SEQUENCE LISTING (8 sequences)

SEQ ID NO: 1 - DNA, 1788 bp, Homo sapiens
atggccgagagtggtgaaagcggcggtcctccgggctcccaggatagcgccgccggagccgaaggtgctggcgcccccgcggccgctgcctccgcggagcccaaaatcatgaaagtcaccgtgaagaccccgaaggaaaaggaggaattcgccgtgcccgagaatagctccgtccagcagtttaaggaagaaatctctaaacgttttaaatcacatactgaccaacttgtgttgatatttgctggaaaaattttgaaagatcaagataccttgagtcagcatggaattcatgatggacttactgttcaccttgtcattaaaacacaaaacaggcctcaggatcattcagctcagcaaacaaatacagctggaacgaatgttactacatcatcaactcctaatagtaactctacatctggttctgctactagcaacccttttggtttaggtggccttgggggacttgcaggtctgagtagcttgggtttgaatactaccaacttctctgaactacagagtcagatgcagcgacaacttttgtctaaccctgaaatgatggtccagatcatggaaaatccctttgttcagagcatgctctcaaatcctgacctgatgagacagttaattatggccaatccacaaatgcagcagttgatacagagaaatccagaaattagtcatatgttgaataatccagatataatgagacaaacgttggaacttgccaggaatccagcaatgatgcaggagatgatgaggaaccaggaccgagctttgagcaacctagaaagcatcccagggggatataatgctttaaggcgcatgtacacagatattcaggaaccaatgctgagtgctgcacaagagcagtttggtggtaatccatttgcttccttggtgagcaatacatcctctggtgaaggtagtcaaccttcccgtacagaaaatagagatccactacccaatccatgggctccacagacttcccagagttcatcagcttccagcggcactgccagcactgtgggtggcactactggtagtactgccagtggcacttctgggcagagtactactgcgccaaatttggtgcctggagtaggagctagtatgttcaacacaccaggaatgcagagcttgttgcaacaaataactgaaaacccacaacttatgcaaaacatgttgtctgccccctacatgagaagcatgatgcagtcactaagccagaatcctgaccttgctgcacagatgatgctgaataatcccctatttgctggaaatcctcagcttcaagaacaaatgagacaacagctcccaactttcctccaacaaatgcagaatcctgatacactatcagcaatgtcaaaccctagagcaatgcaggccttgttacagattcagcagggtttacagacattagcaacggaagccccgggcctcatcccagggtttactcctggcttgggggcattaggaagcactggaggctcttcgggaactaatggatctaacgccacacctagtgaaaacacaagtcccacagcaggaaccactgaacctggacatcagcagtttattcagcagatgctgcaggctcttgctggagtaaatcctcagctacagaatccagaagtcagatttcagcaacaactggaacaactcagtgcaatgggatttttgaaccgtgaagcaaacttgcaagctctaatagcaacaggaggtgatatcaatgcagctattgaaaggttactgggctcccagcatcatagcagcatttctgtatcttga

SEQ ID NO: 2 - protein, 595 aa, Homo sapiens
MAESGESGGPPGSQDSAAGAEGAGAPAAAASAEPKIMKVTVKTPKEKEEFAVPENSSVQQFKEEISKRFKSHTDQLVLIFAGKILKDQDTLSQHGIHDGLTVHLVIKTQNRPQDHSAQQTNTAGTNVTTSSTPNSNSTSGSATSNPFGLGGLGGLAGLSSLGLNTTNFSELQSQMQRQLLSNPEMMVQIMENPFVQSMLSNPDLMRQLIMANPQMQQLIQRNPEISHMLNNPDIMRQTLELARNPAMMQEMMRNQDRALSNLESIPGGYNALRRMYTDIQEPMLSAAQEQFGGNPFASLVSNTSSGEGSQPSRTENRDPLPNPWAPQTSQSSSASSGTASTVGGTTGSTASGTSGQSTTAPNLVPGVGASMFNTPGMQSLLQQITENPQLMQNMLSAPYMRSMMQSLSQNPDLAAQMMLNNPLFAGNPQLQEQMRQQLPTFLQQMQNPDTLSAMSNPRAMQALLQIQQGLQTLATEAPGLIPGFTPGLGALGSTGGSSGTNGSNATPSENTSPTAGTTEPGHQQFIQQMLQALAGVNPQLQNPEVRFQQQLEQLSAMGFLNREANLQALIATGGDINAAIERLLGSQHHSSISVS

SEQ ID NO: 3 - DNA, 2974 bp, Homo sapiens
ggaggaagcggtggctgctgcggatgtcggtgtgagcgagcggcgcctgaacacacggcggctgccgagcgcctgacccgggcctgcgccagagcctgcaccgagctccggggccccacacccgctacggtggccctgcgcccgttgctactgaggcggcgtgctctgcattcttcgctgtccaggcctgccggctctggtgtctgctggctcctccttgctcgcctgctccctcctgcttgcctgagtcaccgccgccgccgccgccacagccatggccgagagtggtgaaagcggcggtcctccgggctcccaggatagcgccgccggagccgaaggtgctggcgcccccgcggccgctgcctccgcggagcccaaaatcatgaaagtcaccgtgaagaccccgaaggaaaaggaggaattcgccgtgcccgagaatagctccgtccagcagtttaaggaagaaatctctaaacgttttaaatcacatactgaccaacttgtgttgatatttgctggaaaaattttgaaagatcaagataccttgagtcagcatggaattcatgatggacttactgttcaccttgtcattaaaacacaaaacaggcctcaggatcattcagctcagcaaacaaatacagctggaagcaatgttactacatcatcaactcctaatagtaactctacatctggttctgctactagcaacccttttggtttaggtggccttgggggacttgcaggtctgagtagcttgggtttgaatactaccaacttctctgaactacagagtcagatgcagcgacaacttttgtctaaccctgaaatgatggtccagatcatggaaaatccctttgttcagagcatgctctcaaatcctgacctgatgagacagttaattatggccaatccacaaatgcagcagttgatacagagaaatccagaaattagtcatatgttgaataatccagatataatgagacaaacgttggaacttgccaggaatccagcaatgatgcaggagatgatgaggaaccaggaccgagctttgagcaacctagaaagcatcccagggggatataatgctttaaggcgcatgtacacagatattcaggaaccaatgctgagtgctgcacaagagcagtttggtggtaatccatttgcttccttggtgagcaatacatcctctggtgaaggtagtcaaccttcccgtacagaaaatagagatccactacccaatccatgggctccacagacttcccagagttcatcagcttccagcggcactgccagcactgtgggtggcactactggtagtactgccagtggcacttctgggcagagtactactgcgccaaatttggtgcctggagtaggagctagtatgttcaacacaccaggaatgcagagcttgttgcaacaaataactgaaaacccacaactgatgcaaaacatgttgtctgccccctacatgagaagcatgatgcagtcactaagccagaatcctgaccttgctgcacagatgatgctgaataatcccctatttgctggaaatcctcagcttcaagaacaaatgagacaacagctcccaactttcctccaacaaatgcagaatcctgatacactatcagcaatgtcaaaccctagagcaatgcaggccttgttacagattcagcagggtttacagacattagcaacggaagccccgggcctcatcccagggtttactcctggcttgggggcattaggaagcactggaggctcttcgggaactaatggatctaacgccacacctagtgaaaacacaagtcccacagcaggaaccactgaacctggacatcagcagtttattcagcagatgctgcaggctcttgctggagtaaatcctcagctacagaatccagaagtcagatttcagcaacaactggaacaactcagtgcaatgggatttttgaaccgtgaagcaaacttgcaagctctaatagcaacaggaggtgatatcaatgcagctattgaaaggttactgggctcccagccatcatagcagcatttctgtatcttgaaaaaatgtaatttatttttgataacggctcttaaactttaaaatacctgctttatttcattttgactcttggaattctgtgctgttataaacaaacccaatatgatgcattttaaggtggagtacagtaagatgtgtgggtttttctgtatttttcttttctggaacagtgggaattaaggctactgcatgcatcacttctgcatttattgtaattttttaaaaacatcaccttttatagttgggtgaccagattttgtcctgcatctgtccagtttatttgctttttaaacattagcctatggtagtaatttatgtagaataaaagcattaaaaagaagcaaatcatttgcactctataatttgtggtacagtattgcttattgtgactttggcatgcatttttgcaaacaatgctgtaagatttatactactgataattttgttttatttgtatacaatatagagtatgcacatttgggactgcatttctggaaacatactgcaataggctctctgagcaaaacacctgtaactaaaaaagtgaagataagaaaatactcttaaagctgagtatttcctaattgtatagaatcttacagcatctttgacaaacatctcccagcaaaagtgccggttagtcaggtttgttgaaaatacagtagaaaagctgattctggttatctctttaaggacaattaattgtacagacacataatgtaacattgtctcaacattcattcacagattgactgtaaattaccttaatctttgtgcagactgaaggaacactgtagtataccccaaagtgcatttgcctaggacttctcagcttctcccataggtagtttaacaggcattaaaatttgtaattgaaatgttgctttcactcaaaaaaaaaaaaaaaaaaaa

SEQ ID NO: 4 - protein, 589 aa, Homo sapiens
MAESGESGGPPGSQDSAAGAEGAGAPAAAASAEPKIMKVTVKTPKEKEEFAVPENSSVQQFKEEISKRFKSHTDQLVLIFAGKILKDQDTLSQHGIHDGLTVHLVIKTQNRPQDHSAQQTNTAGSNVTTSSTPNSNSTSGSATSNPFGLGGLGGLAGLSSLGLNTTNFSELQSQMQRQLLSNPEMMVQIMENPFVQSMLSNPDLMRQLIMANPQMQQLIQRNPEISHMLNNPDIMRQTLELARNPAMMQEMMRNQDRALSNLESIPGGYNALRRMYTDIQEPMLSAAQEQFGGNPFASLVSNTSSGEGSQPSRTENRDPLPNPWAPQTSQSSSASSGTASTVGGTTGSTASGTSGQSTTAPNLVPGVGASMFNTPGMQSLLQQITENPQLMQNMLSAPYMRSMMQSLSQNPDLAAQMMLNNPLFAGNPQLQEQMRQQLPTFLQQMQNPDTLSAMSNPRAMQALLQIQQGLQTLATEAPGLIPGFTPGLGALGSTGGSSGTNGSNATPSENTSPTAGTTEPGHQQFIQQMLQALAGVNPQLQNPEVRFQQQLEQLSAMGFLNREANLQALIATGGDINAAIERLLGSQPS

SEQ ID NO: 5 - protein, 467 aa, Homo sapiens
MTELPAPLSYFQNAQMSEDNHLSNTVRSQNDNREQQDHGDRRRLGNPEPLSNGRPQGNSGPVVERDEEEDEELTLKYGAKHVIMLFVPVTLCMVVVVATIKSVSFYTRKDGQLIYTPFTEDTETVGQRALHSVLNAAIMISVIVVMTILLVVLYKYRCYKVIHAWLIISSLLLLFFFSFIYLGEVFKTYNVAVDYITVALLIWNFGVVGMISIHWKGPLRLQQAYLIMISALMALVFIKYLPEWTAWLILAVISVYDLVAVLCPKGPLRMLVETAQERNETLFPALIYSSTMVWLVNMAEGDPEAQRRVSKNTKYNAQGTEREAQASVPENDDGGFSEEWEAQRDSQLGPHRSTSVSRAAVQEISSSIPASEDPEERGVKLGLGDFVFYSVLVGKASATASGDWNTTIACFVAILIGLCLTLLLLAIFKKALPALPISITFGLVFYFATDYLVQPFMDQLAFHQFYI

SEQ ID NO: 6 - protein, 448 aa, Homo sapiens
MLTFMASDSEEEVCDERTSLMSAESPTPRSCQEGRQGPEDGENTAQWRSQENEEDGEEDPDRYVCSGVPGRPPGLEEELTLKYGAKHVIMLFVPVTLCMIVVVATIKSVRFYTEKNGQLIYTPFTEDTPSVGQRLLNSVLNTLIMISVIVVMTIFLVVLYKYRCYKFIHGWLIMSSLMLLFLFTYIYLGEVLKTYNVAMDYPTLLLTVWNFGAVGMVCIHWKGPLVLQQAYLIMISALMALVFIKYLPEWSAWVILGAISVYDLVAVLCPKGPLRMLVETAQERNEPIFPALIYSSAMVWTVGMAKLDPSSQGALQLPYDPEMEEDSYDSFGEPSYPEVFEPPLTGYPGEELEEEEERGVKLGLGDFIFYSVLVGKAAATGSGDWNTTLACFVAILIGLCLTLLLLAVFKKALPALPISITFGLIFYFSTDNLVRPFMDTLASHQLYI

SEQ ID NO: 7 - DNA, 2763 bp, Homo sapiens
tgggacaggcagctccggggtccgcggtttcacatcggaaacaaaacagcggctggtctggaaggaacctgagctacgagccgcggcggcagcggggcggcggggaagcgtatacctaatctgggagcctgcaagtgacaacagcctttgcggtccttagacagcttggcctggaggagaacacatgaaagaaagaacctcaagaggctttgttttctgtgaaacagtatttctatacagttgctccaatgacagagttacctgcaccgttgtcctacttccagaatgcacagatgtctgaggacaaccacctgagcaatactgtacgtagccagaatgacaatagagaacggcaggagcacaacgacagacggagccttggccaccctgagccattatctaatggacgaccccagggtaactcccggcaggtggtggagcaagatgaggaagaagatgaggagctgacattgaaatatggcgccaagcatgtgatcatgctctttgtccctgtgactctctgcatggtggtggtcgtggctaccattaagtcagtcagcttttatacccggaaggatgggcagctaatctataccccattcacagaagataccgagactgtgggccagagagccctgcactcaattctgaatgctgccatcatgatcagtgtcattgttgtcatgactatcctcctggtggttctgtataaatacaggtgctataaggtcatccatgcctggcttattatatcatctctattgttgctgttctttttttcattcatttacttgggggaagtgtttaaaacctataacgttgctgtggactacattactgttgcactcctgatctggaattttggtgtggtgggaatgatttccattcactggaaaggtccacttcgactccagcaggcatatctcattatgattagtgccctcatggccctggtgtttatcaagtacctccctgaatggactgcgtggctcatcttggctgtgatttcagtatatgatttagtggctgttttgtgtccgaaaggtccacttcgtatgctggttgaaacagctcaggagagaaatgaaacgctttttccagctctcatttactcctcaacaatggtgtggttggtgaatatggcagaaggagacccggaagctcaaaggagagtatccaaaaattccaagtataatgcagaaagcacagaaagggagtcacaagacactgttgcagagaatgatgatggcgggttcagtgaggaatgggaagcccagagggacagtcatctagggcctcatcgctctacacctgagtcacgagctgctgtccaggaactttccagcagtatcctcgctggtgaagacccagaggaaaggggagtaaaacttggattgggagatttcattttctacagtgttctggttggtaaagcctcagcaacagccagtggagactggaacacaaccatagcctgtttcgtagccatattaattggtttgtgccttacattattactccttgccattttcaagaaagcattgccagctcttccaatctccatcacctttgggcttgttttctactttgccacagattatcttgtacagccttttatggaccaattagcattccatcaattttatatctagcatatttgcggttagaatcccatggatgtttcttctttgactataacaaaatctggggaggacaaaggtgattttcctgtgtccacatctaacaaagtcaagattcccggctggacttttgcagcttccttccaagtcttcctgaccaccttgcactattggactttggaaggaggtgcctatagaaaacgattttgaacatacttcatcgcagtggactgtgtccctcggtgcagaaactaccagatttgagggacgaggtcaaggagatatgataggcccggaagttgctgtgccccatcagcagcttgacgcgtggtcacaggacgatttcactgacactgcgaactctcaggactaccgttaccaagaggttaggtgaagtggtttaaaccaaacggaactcttcatcttaaactacacgttgaaaatcaacccaataattctgtattaactgaattctgaacttttcaggaggtactgtgaggaagagcaggcaccagcagcagaatggggaatggagaggtgggcaggggttccagcttccctttgattttttgctgcagactcatcctttttaaatgagacttgttttcccctctctttgagtcaagtcaaatatgtagattgcctttggcaattcttcttctcaagcactgacactcattaccgtctgtgattgccatttcttcccaaggccagtctgaacctgaggttgctttatcctaaaagttttaacctcaggttccaaattcagtaaattttggaaacagtacagctatttctcatcaattctctatcatgttgaagtcaaatttggattttccaccaaattctgaatttgtagacatacttgtacgctcacttgccccagatgcctcctctgtcctcattcttctctcccacacaagcagtctttttctacagccagtaaggcagctctgtcgtggtagcagatggtcccattattctagggtcttactctttgtatgatgaaaagaatgtgttatgaatcggtgctgtcagccctgctgtcagaccttcttccacagcaaatgagatgtatgcccaaagcggtagaattaaagaagagtaaaatggctgttgaagc

SEQ ID NO: 8 - DNA, 2236 bp, Homo sapiens
cgagcggcggcggagcaggcatttccagcagtgaggagacagccagaagcaagctattggagctgaaggaacctgagacagaagctagtcccccctctgaattttactgatgaagaaactgaggccacagagctaaagtgacttttcccaaggtcgcccagcgaggacgtgggacttctcagacgtcaggagagtgatgtgagggagctgtgtgaccatagaaagtgacgtgttaaaaaccagcgctgccctctttgaaagccagggagcatcattcatttagcctgctgagaagaagaaaccaagtgtccgggattcagacctctctgcggccccaagtgttcgtggtgcttccagaggcagggctatgctcacattcatggcctctgacagcgaggaagaagtgtgtgatgagcggacgtccctaatgtcggccgagagccccacgccgcgctcctgccaggagggcaggcagggcccagaggatggagagaacactgcccagtggagaagccaggagaacgaggaggacggtgaggaggaccctgaccgctatgtctgtagtggggttcccgggcggccgccaggcctggaggaagagctgaccctcaaatacggagcgaagcacgtgatcatgctgtttgtgcctgtcactctgtgcatgatcgtggtggtagccaccatcaagtctgtgcgcttctacacagagaagaatggacagctcatctacacgacattcactgaggacacaccctcggtgggccagcgcctcctcaactccgtgctgaacaccctcatcatgatcagcgtcatcgtggttatgaccatcttcttggtggtgctctacaagtaccgctgctacaagttcatccatggctggttgatcatgtcttcactgatgctgctgttcctcttcacctatatctaccttggggaagtgctcaagacctacaatgtggccatggactaccccaccctcttgctgactgtctggaacttcggggcagtgggcatggtgtgcatccactggaagggccctctggtgctgcagcaggcctacctcatcatgatcagtgcgctcatggccctagtgttcatcaagtacctcccagagtggtccgcgtgggtcatcctgggcgccatctctgtgtatgatctcgtggctgtgctgtgtcccaaagggcctctgagaatgctggtagaaactgcccaggagagaaatgagcccatattccctgccctgatatactcatctgccatggtgtggacggttggcatggcgaagctggacccctcctctcagggtgccctccagctcccctacgacccggagatggaagaagactcctatgacagttttggggagccttcataccccgaagtctttgagcctcccttgactggctacccaggggaggagctggaggaagaggaggaaaggggcgtgaagcttggcctcggggacttcatcttctacagtgtgctggtgggcaaggcggctgccacgggcagcggggactggaataccacgctggcctgcttcgtggccatcctcattggcttgtgtctgaccctcctgctgcttgctgtgttcaagaaggcgctgcccgccctccccatctccatcacgttcgggctcatcttttacttctccacggacaacctggtgcggccgttcatggacaccctggcctcccatcagctctacatctgagggacatggtgtgccacaggctgcaagctgcagggaattttcattggatgcagttgtatagttttacactctagtgccatatatttttaagacttttctttccttaaaaaataaagtacgtgtttacttggtgaggaggaggcagaaccagctctttggtgccagctgtttcatcaccagactttggctcccgctttggggagcgcctcgcttcacggacaggaagcacagcaggtttatccagatgaactgagaaggtcagattagggcggggagaagagcatccggcatgagggctgagatgcgcaaagagtgtgctcgggagtggcccctggcacctgggtgctctggctggagaggaaaagccagttccctacgaggagtgttcccaatgctttgtccatgatgtccttgttattttattgcctttagaaactgagtcctgttcttgttacggcagtcacactgctgggaagtggcttaatagtaatatcaataaatagatgagtcctgttagaaaaa

(19) United States
(12) Patent Application Publication
(10) Pub. No.: US 2003/0175278 A1
(43) Pub. Date: Sep. 18, 2003

(54) Ubiquilin, a presenilin interactor and methods of using same

(76) Inventors: Mervyn J. Monteiro, Columbia, MD (US); Alex L. Mah, Baltimore, MD (US); George Perry, University Heights, OH (US); Mark A. Smith, Cleveland, OH (US)

Correspondence Address: INTELLECTUAL PROPERTY / TECHNOLOGY LAW, PO BOX 14329, RESEARCH TRIANGLE PARK, NC, 27709, US

(21) Appl. No.: 10/293,000

(22) Filed: Nov. 13, 2002

Related U.S. Application Data
(60) Provisional application No. 60/338,549, filed on Nov. 13, 2001.

(51) Int. Cl. A61K 39/395; G01N 33/53; G01N 33/567; C07H 21/04; C12N 9/64; C12P 21/02; C12N 5/06; C07K 16/40

(52) U.S. Cl. 424/146.1; 435/7.2; 435/69.1; 435/320.1; 435/325; 435/226; 536/23.2; 530/388.26

(57) ABSTRACT

The present invention describes a human presenilin-interacting protein, ubiquilin, that modulates presenilin protein levels and is associated with neuropathological lesions such as neurofibrillary tangles and Lewy bodies in Alzheimer's disease and Parkinson's disease affected brains. Also, the present invention relates to anti-ubiquilin antibodies that prominently stain neurons. The present invention further relates to introducing and expressing ubiquilin to modulate cell functions relating to presenilin synthesis.

### CROSS REFERENCE TO RELATED APPLICATIONS

This application claims the benefit from U.S. Provisional Application Serial No. 60/338,549, filed Nov. 13, 2001.

### BACKGROUND OF THE INVENTION

1. Field of the Invention

The present invention generally relates to protein-protein interaction, and more particularly, to a presenilin interactor protein, hereinafter referred to as ubiquilin, which modulates presenilin activity.

2. Background of the Related Art

Alzheimer's disease (AD) is a progressive neurodegenerative disorder characterized by impaired memory and cognition, as well as altered behavior. The majority of AD cases are late-onset, appearing in people over the age of 65. However, a small percent (5%) of cases, termed early-onset, arise at an unusually young age, as early as the third decade of life. Molecular genetic analysis has linked early-onset familial Alzheimer's disease (FAD) to the autosomal dominant inheritance of mutations in three genes: the β-amyloid precursor protein (APP) and two homologous genes, presenilin-1 and -2 (PS1 and PS2) (Hardy 1997; Price, et al. 1998). The mechanisms by which mutations in these three genes cause AD are unresolved.^{N}

PS1 and PS2 are multitransmembrane proteins that share 67% sequence identity. The topology of presenilins is debatable, though the most widely drawn models show proteins that weave through the membrane eight times, with the NH₂— and COOH-terminal domains and a large “loop” between transmembrane domains six and seven all oriented towards the cytoplasm (see FIG. 1A) (Haass and De Strooper 1999). Although both proteins share extensive sequence identity along their entire lengths, their NH₂-terminal domains and the second half of their loops are highly divergent, suggesting that these unique regions could modulate different functions of the two presenilins. An examination of presenilin-linked FAD mutations mapped so far reveals that most are missense mutations in identical residues spread throughout the polypeptide in both presenilins, suggesting that the conserved residues are important for the function of the proteins.

Studies of presenilin homologues in various species have indicated that presenilin genes are required for proper development. Mutation of the Caenorhabditis elegans presenilin gene, sel-12, produces defects in vulva development linked to cell signaling defects involving Notch-based receptors (Levitan and Greenwald 1995; Haass and De Strooper 1999). Disruption of the Drosophila melanogaster presenilin gene results in embryonic lethality, with embryos displaying severe Notch-like phenotypes (Struhl and Greenwald 1999; Ye, et al. 1999). Mammalian presenilins also play important roles in early development, as disruption of the mouse PS1 gene leads to death shortly after birth, with embryos displaying central nervous system defects together with abnormal patterning of the axial skeleton and spinal ganglia (Shen, et al. 1997; Wong, et al. 1997). Interestingly, PS1^{-/-} mice can be rescued by a human transgenes containing FAD-linked mutations, indicating that the FAD mutations do not affect presenilin functions related to embryo development (Davis, et al. 1998; Qian, et al. 1998). In contrast, disruption of the mouse PS2 gene produces no obvious defects, but PS1/PS2 double knock-out mice die earlier at embryonic day 9.5 and, like PS1^{-/-} mice, display severe misexpression of proteins involved in Notch signaling (Donoviel, et al. 1999; Herreman, et al. 1999). These results indicate that there is functional redundancy between PS1 and PS2; PS1 can compensate for PS2, but PS2 cannot compensate for PS1, at least during early mouse development.

Both human presenilin genes are ubiquitously expressed, but at low levels. In the brain, the proteins are more highly expressed in neurons than glia (Price, et al. 1998). Knowledge of where presenilin proteins localize in cells is important for understanding their function. However, uncertainties exist regarding their precise subcellular localization. In neurons, endogenous PS1 and PS2 have been localized to the ER and to vesicular structures of the somatodentritic compartment and axons (Price, et al. 1998; Haass and De Strooper 1999).

When overexpressed, the proteins predominantly localize to the ER, the Golgi complex, and the nuclear envelope (Kovacs,,. 1996; Janicki and Monteiro 1997; Haass and De Strooper 1999). However, in nonneuronal cells, endogenous presenilins have been localized to the ER, Golgi complex, centrosomes, centromeres, and cell surface (Georgakopoulos, et al. 1999; Haass and De Strooper 1999; Raina et al. 1999). It is not known to what extent these diverse locations reflect true sites of presenilin function.

The presenilin proteins have been linked to several cellular functions. Interestingly, some of these cellular functions are compromised or altered by the expression of PS genes containing FAD mutations. The presenilin proteins have been shown to play important roles in apoptosis, calcium homeostasis, cell cycle regulation, regulation of misfolded proteins in the ER, and cleavage of APP (Guo, et al. 1997; Janicki and Monteiro 1997, Janicki and Monteiro 1999; Cotman 1998; Mattson et al. 1998; Haass and De Strooper 1999; Katayama, et al. 1999; Leissring, et al. 1999; Niwa, et al. 1999). Further clues regarding presenilin functions are emerging from a growing list of proteins with which presenilin interacts. Previously, a newly discovered calcium-binding protein, which preferentially interacts with the PS2 loop and modulates presenilin-induced cell death was identified and characterized as “calmyrin” (Stabler, et al. 1999). Thus, it would be advantageous to identify other proteins that interact and/or bind to the expressed presenilin proteins therebv linking these proteins to known pathways or structures that modulate the activity of normal and mutant presenilins.

### SUMMARY OF THE INVENTION

The present invention relates to identifying a human presenilin-interacting protein, ubiquilin, that modulates presenilin protein levels and is associated with neuropathological lesions such as neurofibrillary tangles (NFTs) and Lewy bodies in AD and Parkinson's disease (PD) affected brains.

In one aspect, the present invention provides for a purified and isolated nucleic acid molecule encoding a protein that modulates presenilin protein levels having the amino acid sequence of SEQ ID NO: 2.

In another aspect, the invention relates to an isolated and purified nucleic acid molecule having the nucleic acid sequence of SEQ ID NO: 1.

The present invention also provides a method for producing a polypeptide comprising an amino acid sequence of SEQ ID NO: 2, the method comprising:

(a) culturing a host cell with an expression vector containing a polynucleotide sequence of SEQ ID NO: 1; and

(b) recovering the expressed polypeptide from the host cell line.

The present invention further relates to purified antibodies, including polyclonal and monoclonal, that bind to a polypeptide comprising an amino acid sequence as set forth in SEQ ID NO: 2 which is useful in diagnostic methods.

In another aspect, the invention relates to a vector for transforming a mammalian tissue cell to express effective amounts of ubiquilin having the amino acid residues of SEQ ID NO: 2 or SEQ ID NO. 4. Preferably, another protein of choice is coexpressed with the ubiquilin, wherein the protein of choice may be presenilin 1 or 2 having amino acid residues of SEQ ID NO. 5 and SEQ ID NO. 6, respectively. The vector may be delivered to the cells by a suitable vehicle, including but not limited to vaccinia virus, adeno associated virus, retrovirus, liposome transport, neuraltropic viruses and other vector systems.

In a still further aspect, the invention relates to a method of increasing accumulation of presenilin 1 and/or 2 by expressing ubiquilin, a protein having the ability to modulate synthesis activity of presenilin 1 and 2, the method comprising:

introducing an expression vector to a host cell that expresses presenilin 1 and/or 2, wherein the expression vector comprises a nucleotide sequence encoding an amino acid sequence of SEQ ID NO. 2 or SEQ ID NO. 4;

maintaining the transformed host cell under biological conditions sufficient for expression and accumulation of the ubiquilin in the host cell; and

measuring the increase of presenilin 1 and/2 in the host cell.

In yet another aspect, the invention relates to an assay method to determine the presence of ubiquilin and presenilin 1 and/or 2 that is associated with neuropathological lesions, such as neurofibrillary tangles (NFTs) and Lewy bodies in Alzheimer's disease and Parkinson's disease affected brains, wherein the immunoassay utilizes an antibody against ubiquilin, the method comprising:

contacting sample brain tissue with an antibody, which specifically binds to the ubiquilin to form a mixture of antigen-antibody reaction products;

detecting the presence of the antigen-antibody reaction products; and

correlating the detected antigen-antibody reaction products with a control standard to show presence of presenilin 1 and/or 2 in brain tissue.

The present invention further provides for a method to modulate presenilin protein levels by protein-protein interaction, the method comprising:

interacting a presenilin peptide comprising an amino acid sequence of SEQ ID NO. 5 or SEQ ID NO 6 with a ubiquilin protein of SEQ ID NO. 2 or SEQ ID NO. 4.

The present invention further provides for a method of quantifying neurofibrillary tangles and Lewy bodies in Alzheimer's disease and Parkinson's disease affected brains, the method comprising:

contacting sample brain tissue with an anti-ubiquilin antibody, which specifically binds to the ubiquilin to form a mixture of antigen-antibody reaction products;

detecting the presence of the antigen-antibody reaction products; and

correlating the detected antigen-antibody reaction products with a control standard to show and quantify neurofibrillary tangles and Lewy bodies in the brain tissue.

Preferably the anti-ubiquilin antibody is detected by an indicator affixed to the antibody. Exemplary and well known such indicators include radioactive labels (e.g., ³²P, ¹²⁵I, ¹⁴C), a second antibody or an enzyme such as horse radish peroxidase.

Other features and advantages of the invention will be apparent from the following detailed description, drawings and claims.

### BRIEF DESCRIPTION OF THE FIGURES

FIG. 1A is a schematic diagram of ER-bound human PS2 and shows eight transmembrane domains with its NH₂ terminus, large hydrophilic loop, and COOH terminus all protruding into the cytoplasm. PS1 is believed to have a similar structure.

FIG. 1B shows the amino acid sequence of PS1 and PS2 COOH terminus and loop regions that were used as Y2H baits.

FIG. 1C shows the results of Y2H β-galactosidase liquid culture interaction assay of an ubiquilin-prey clone (FIG. 2 III, construct B without GST) with PS2-COOH terminus (PS2-C), LexA alone, nuclear lamin B, and CENP-C baits. The β-galactosidase units were normalized to CENP-C, the bait with the weakest interaction.

FIG. 1D shows the results of β-Galactosidase interaction assay data of a near full-length ubiquilin-prey clone (FIG. 2 III, construct H without GST) repeated with the same baits described in C along with PS1-COOH (PS1-C) terminus, PS2-Loop (PS2-L), and PS1-Loop (PS1-L) baits. Again, β-galactosidase units were normalized to CENP-C

FIG. 2 illustrates schematic drawings of ubiquilin expression constructs.

(I) The full-length ubiquilin polypeptide consists of 595 residues and contains an NH2— terminal UB domain (speckled), a COOH-terminal UBA domain (striped), and several regularly spaced asparagine-proline (Asn-Pro) repeats (vertical bars).

(II) The probes used in human Northern blots.

(III) GST-fusion constructs: A (N393-S595 aa), B (Q378-S595 aa), C (Q113-M377 aa), D (Q541-S595 aa), E (D449-S595 aa), F (D449-L540 aa), G (N393-L540 aa), H (M37-S595 aa), I (M37-L540 aa), J (Q113-L540 aa), K (Q113-S595 aa), and L (GST alone). The ubiquilin portions of constructs A and B were isolated in the original Y2H screen. Bacterially expressed GST-fusion B and C polypeptides were used as immunogens for anti-ubiquilin pAb production, as shown in FIG. 3.

(IV) Mammalian expression constructs: M, full-length untagged ubiquilin; N, NH₂- terminal GFP-tagged ubiquilin fused at residue 20 (Ala); and O, COOH-terminal myc epitope-tagged ubiquilin.

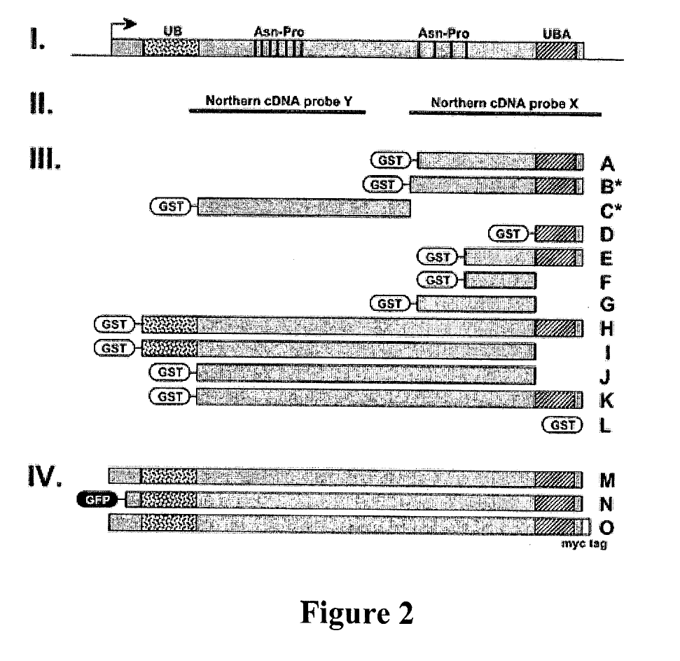

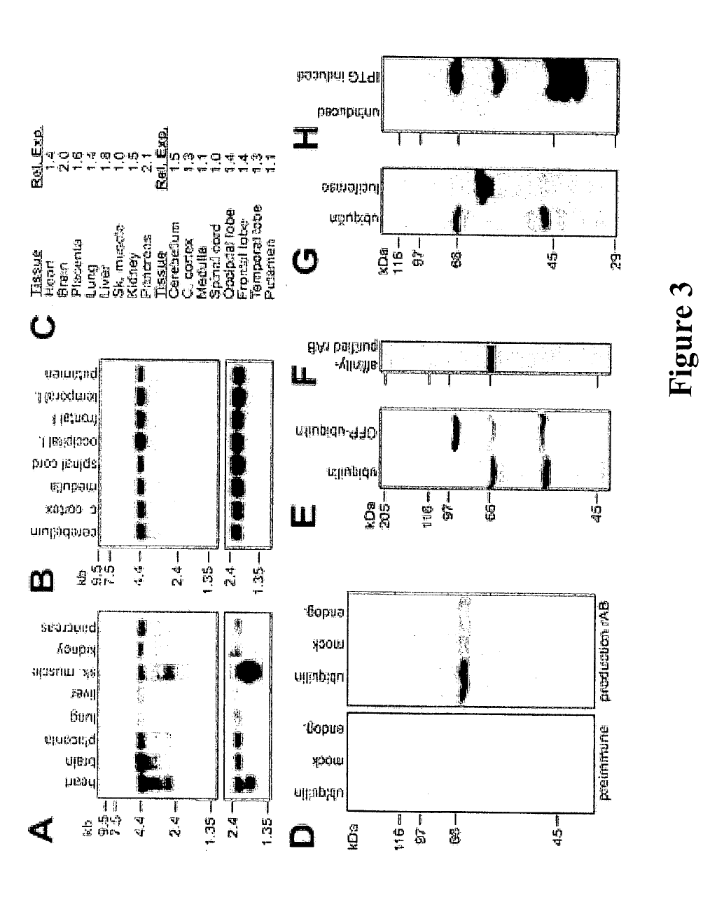

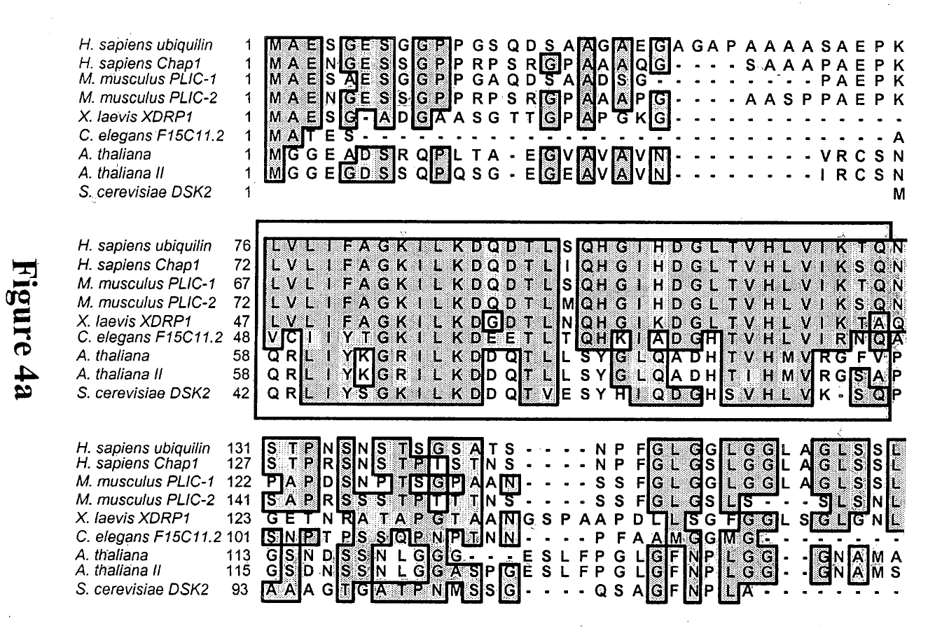

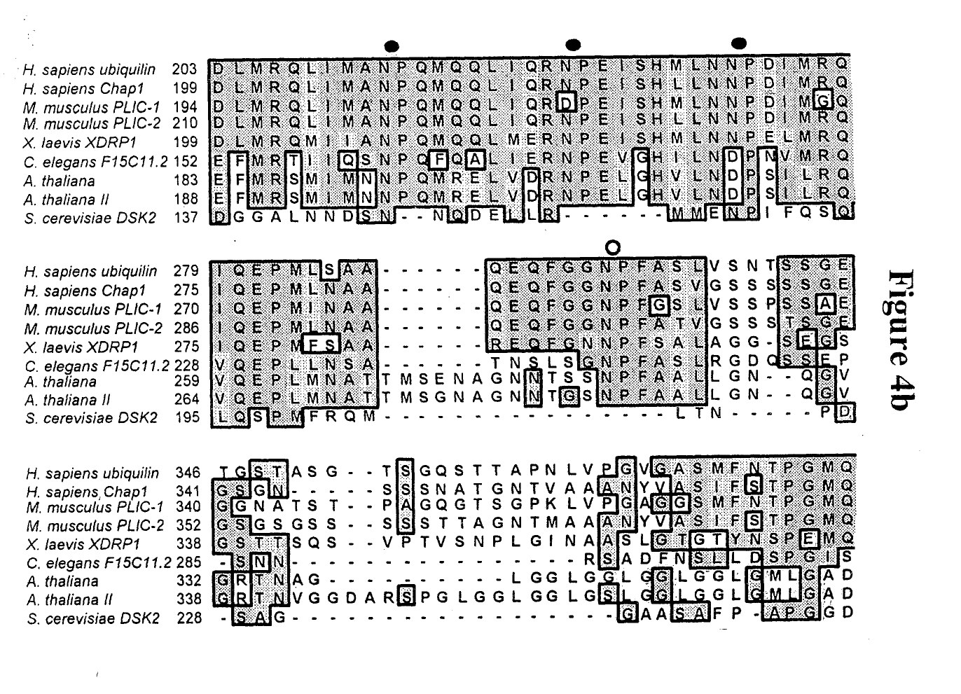

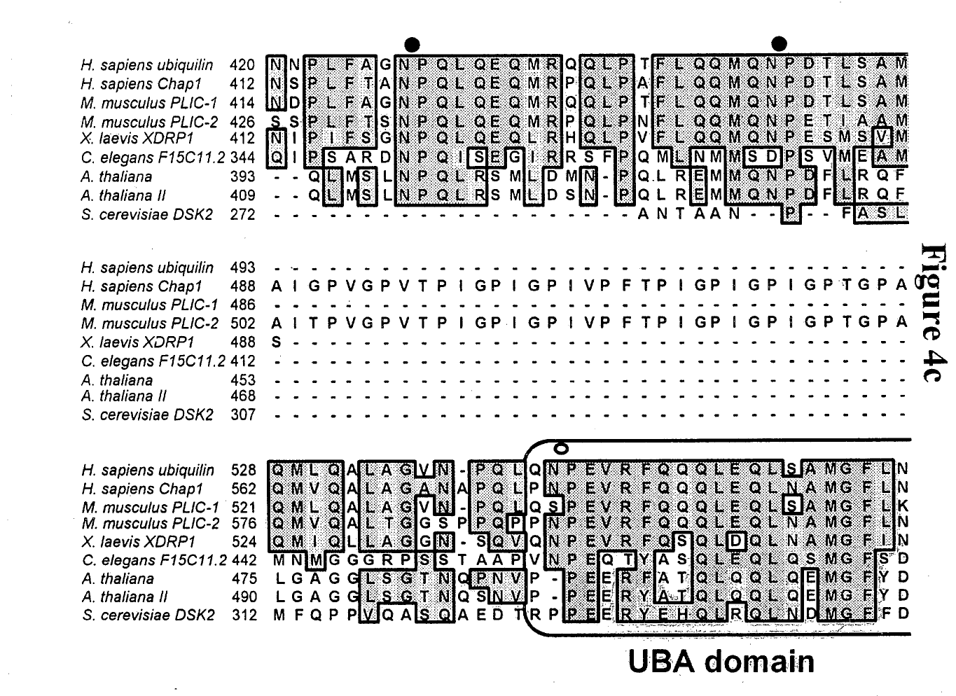

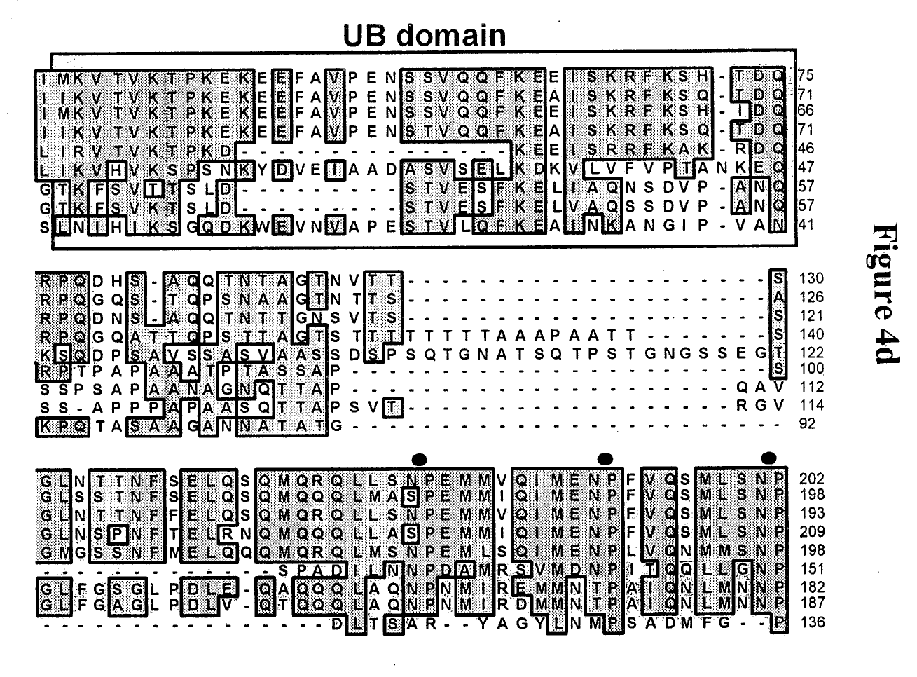

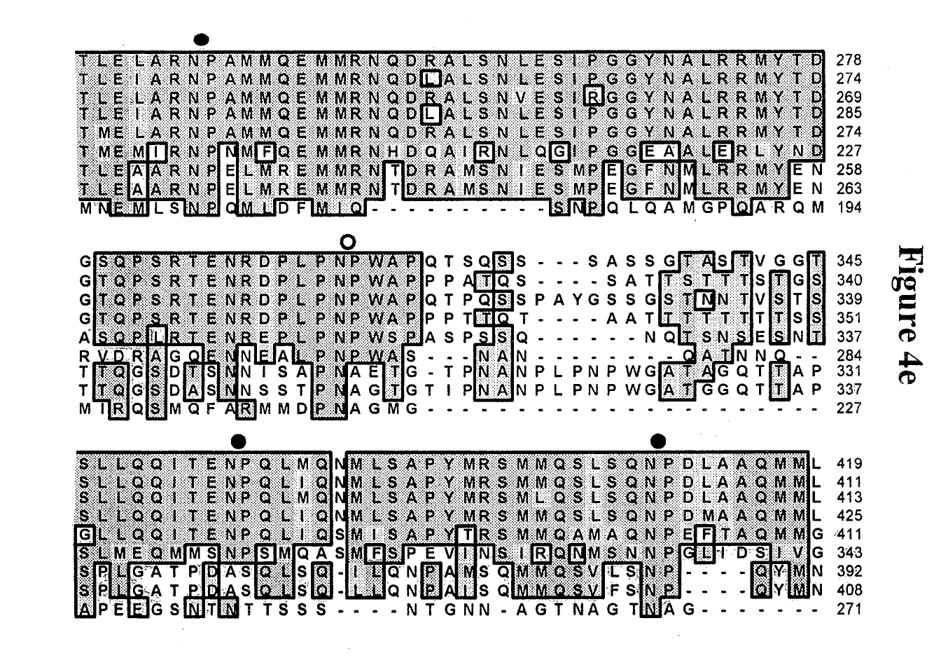

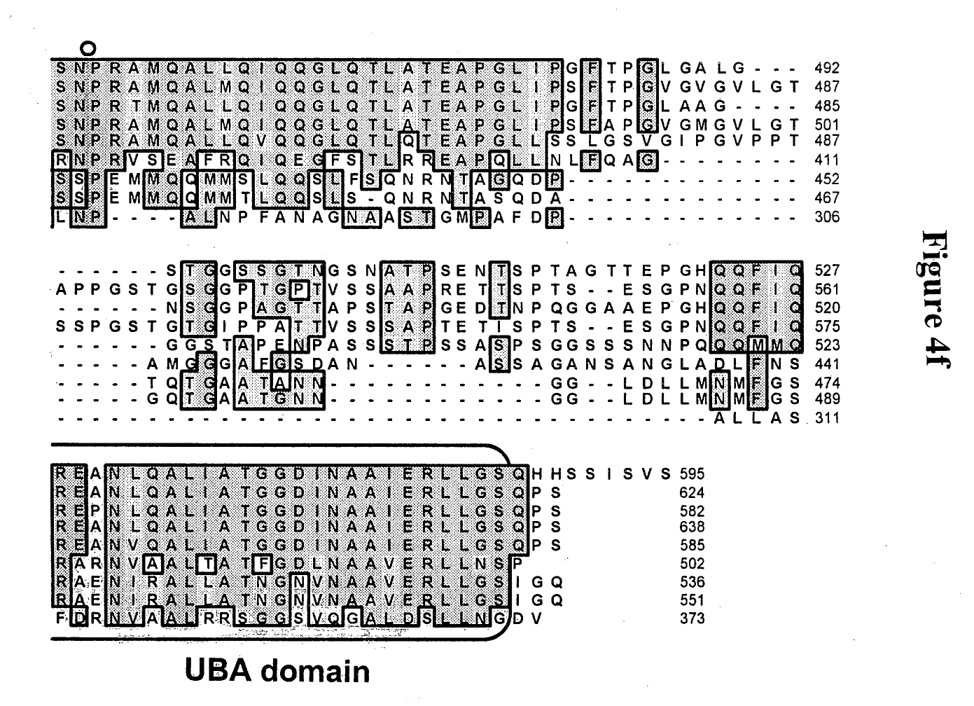

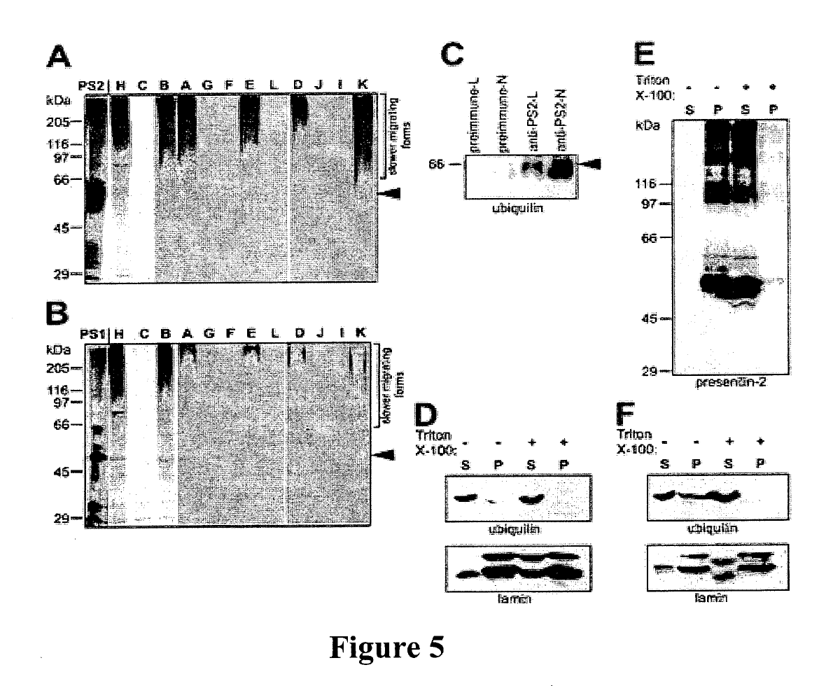

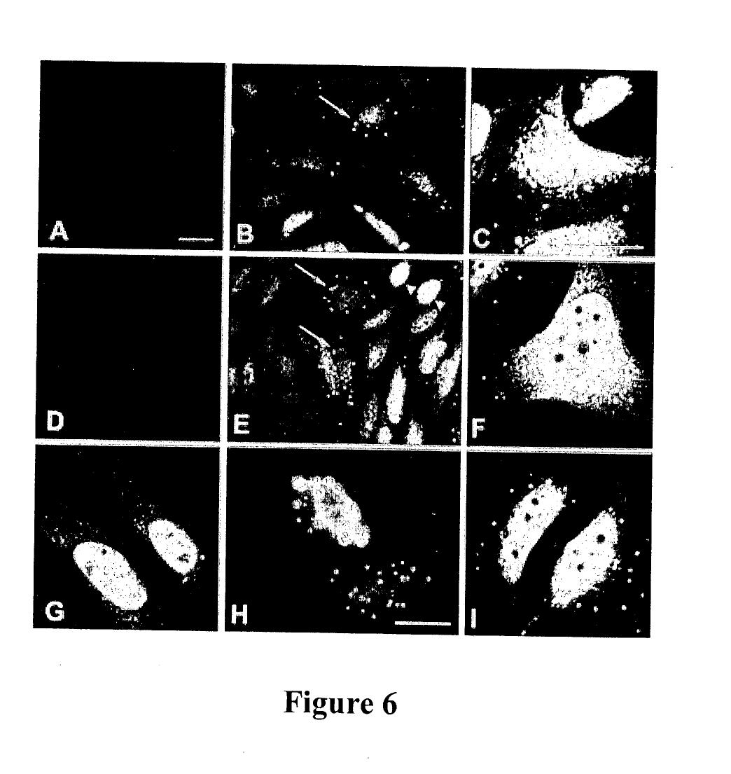

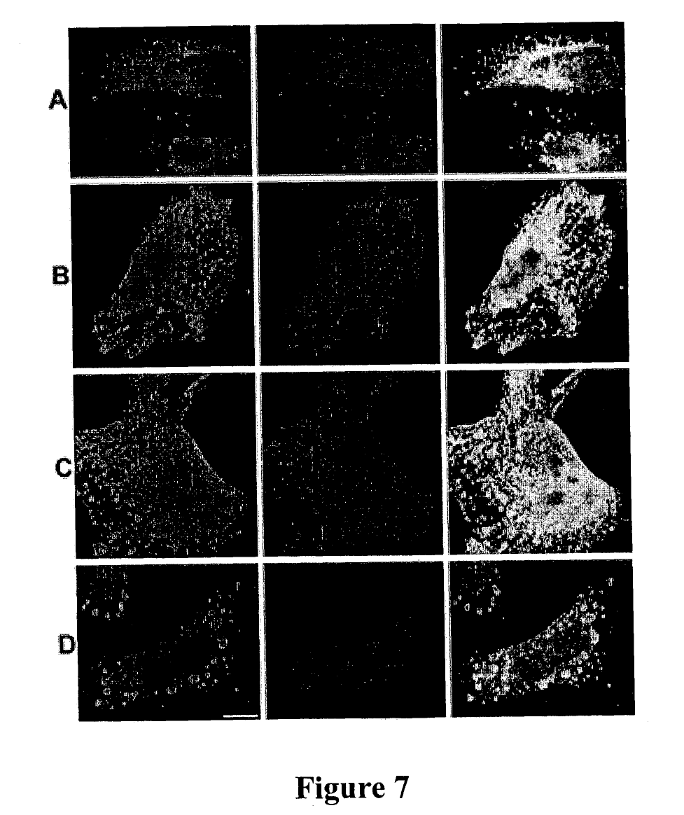

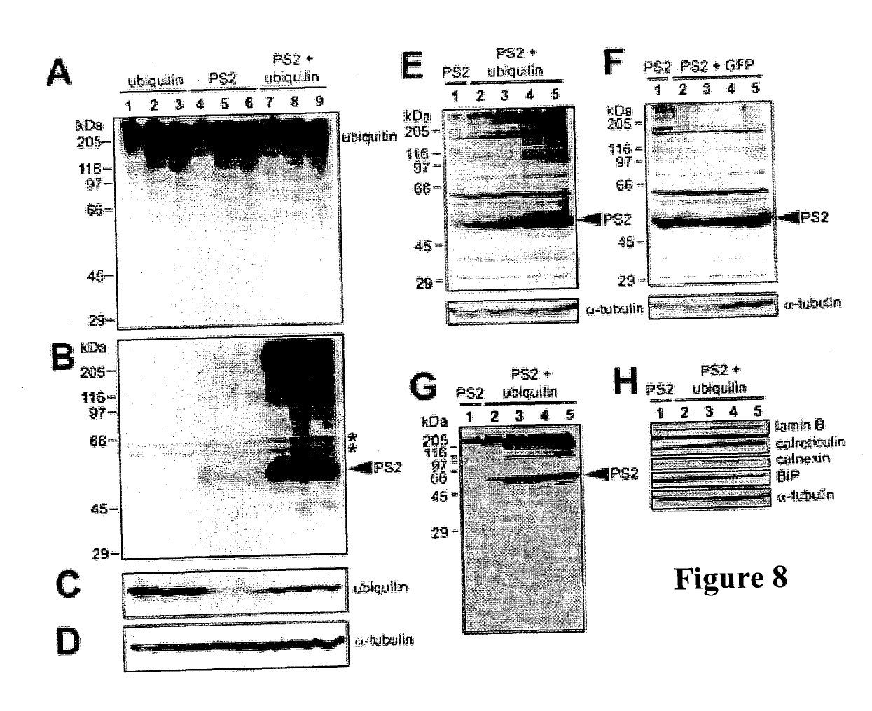

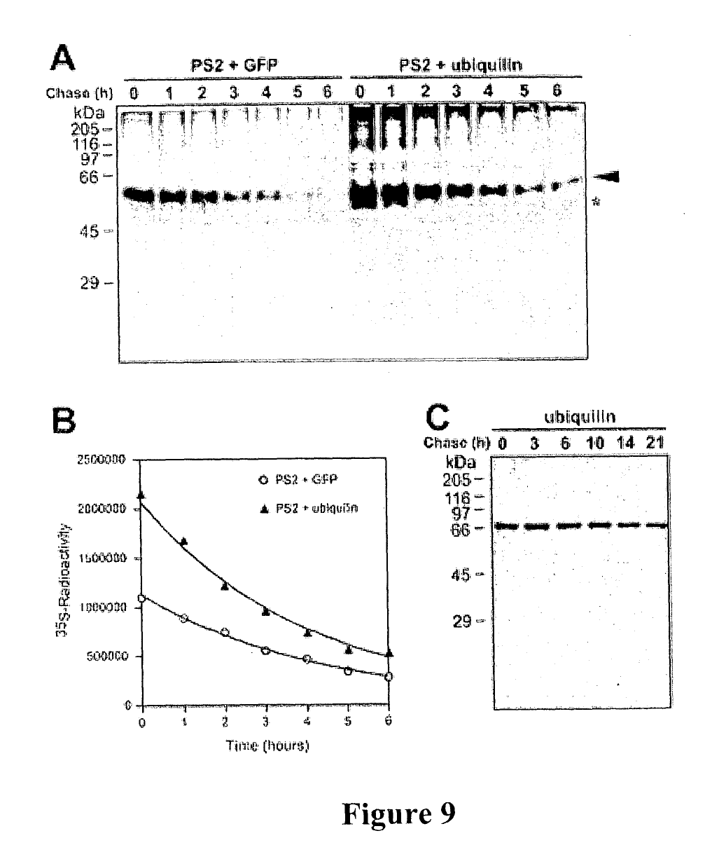

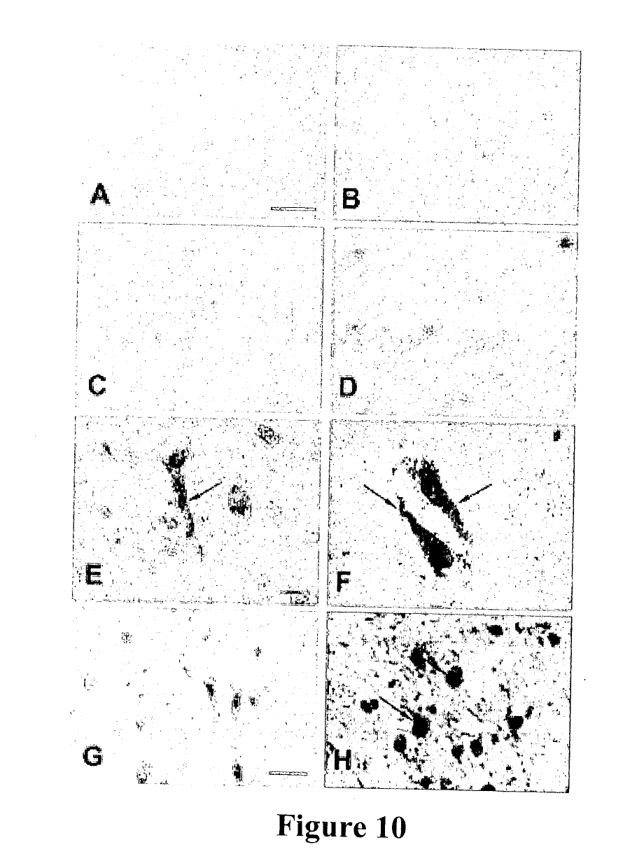

FIG. 3A shows the immunoblot of multiple human tissue probed with ubiquilin cDNA fragment X (FIG. 2 II). After stripping, the blot was reprobed with a β-actin control fragment.

FIG. 3B shows the Northern blot of specific regions of the human brain probed with ubiquilin cDNA fragment Y (FIG. 2 II), with a reprobe with β-actin, which is shown below.

FIG. 3C compiles the results of quantification of ubiquilin mRNA expression levels. Relative expression of ubiquilin in different tissues was determined by densitometric analysis of the autoradiographs and relating the ubiquilin band intensities to the levels of β-actin hybridization from the same lanes. The values are presented after normalization against skeletal muscle (above) and spinal cord (below).

FIG. 3D shows that the anti-ubiquilin-B antibody detected a 66-kD doublet band, whereas preimmune sera did not.

FIG. 3E shows that the anti-ubiquilin-C antibody also recognized the 66-kD band in untransfected HeLa lysates and to varying extents a 55-kD band. HeLa cells transfected with GFP-ubiquilin (FIG. 2 IV, N) contain an additional 93-kD reactive band, due to the fusion of the 27-kD moiety of GFP with ubiquilin.

FIG. 3F shows that the affinity-purified anti-ubiquilin-C antibody specifically reacts with the 66-kD band from transfected HeLa cell lysates.

FIG. 3G shows that the full-length in vitro transcribed and translated [³⁵S]methionine-radiolabeled human ubiquilin polypeptides migrated at 66 kD, whereas radiolabeled luciferase migrated at 61 kD.

FIG. 3H shows the uninduced and IPTG-induced lysates of bacteria transformed with untagged full-length human ubiquilin and probed with anti-ubiquilin-B antibodies. Full-length immunoreactive ubiquilin (66 kD) and a series of smaller breakdown products are only seen in the induced lysate.

FIG. 4 illustrates that ubiquilin shares significant homology with several other proteins. The inferred amino acid sequence of the human ubiquilin ORF and its homology to several related proteins: Homo sapiens Chap1, a protein involved in binding Hsp7O-like Stch protein; Mus musculus PLIC-1 and PLIC-2, proteins involved in linking integrins to vimentin; X. laevis XDRP1, a protein which binds cyclin A; C. elegans F15C11.2 and two A. thaliana proteins, proteins with unknown functions; and S. cerevisiae DSK2, involved in spindle pole body duplication. Identical residues are darkly shaded, whereas similar residues are lightly shaded. Only homology in five or more sequences are shown. All the proteins contain UB domains (square box) and UBA domains (rounded box), which are extremely well conserved in sequence. In addition, the central region between the UB and UBA domains contain several asparagine-proline repeats, which are either regularly spaced apart (closed circles) or located elsewhere throughout the protein (open circles).

FIGS. 5A and B show GST pull-down experiments. Full-length in vitro synthesized ³⁵S-labeled PS2 and PS1 (first lanes) migrated in SDS-PAGE gels with broad bands of 54 and 48 kD (arrowheads), respectively, along with a smear of slower migrating forms, presumably due to the highly hydrophobic nature of the proteins. [³⁵S]PS complexes (especially the slower migrating forms) were retained by GST-ubiquilin constructs containing the UBA domain (lane letters correspond to constructs shown in FIG. 2 III), but not by those lacking the domain, or by GST alone.

FIG. 5C shows the results of PS2-transfected HeLa cell lysates that were immunoprecipitated with preimmune sera or corresponding anti-PS2-Loop antibody and anti-PS2 NH² terminus antibody. After separation by SDS-PAGE, coprecipitating ubiquilin (arrowhead) was detected by immunoblotting with anti-ubiquilin-B antibody.

FIGS. 5D-5F show the results of cell fractionation experiments. Parallel immunoblots of equal portions of soluble supernatant (S) and insoluble pellet (P) HeLa cellular fractions were prepared without the use of detergent (−) or in the presence of 1% Triton X-100 (+) with (D and F) anti-ubiquilin or (E) anti-PS2 antibodies. The HeLa cells used for cell fractionation in D were untransfected, whereas, in E and F, the cells were transfected with a full-length wild-type PS2 construct. The relative ratio of ubiquilin protein in the P-compared with the S-fractions was determined by densitometric analysis of the autoradiographs. This analysis indicated that transfection of presenilin caused 30% more (F) ubiquilin protein to partition in the pellet fraction in the absence of detergent compared with (D) untransfected cells. The partitioning of lamin A/C proteins after cell fractionation from untransfected and PS2-transfected cells was monitored with anti-lamin A/C antibodies and shown below in D and F, respectively.

FIGS. 6A and B show preimmune and the corresponding anti-ubiquilin-B antibody staining, respectively.

FIG. 6C shows overexpressed ubiquilin as detected with anti-ubiquilin-B antibody.

FIGS. 6D and E show preimmune and the corresponding anti-ubiquilin-C antibody staining, respectively.

FIG. 6F shows overexpressed ubiquilin, as detected with affinity-purified anti-ubiquilin-C antibody. Both anti-ubiquilin sera showed specific staining in the cytoplasm and nucleus, along with cytoplasmic punctate structures in a subset of the untransfected cells (arrows). The expression levels of ubiquilin protein within the nucleus varied with some cells containing substantially more nuclear protein (arrowheads). Transient overexpression of wild-type ubiquilin caused frequent accumulation of ubiquilin to the intracellular punctate structures.

FIG. 6G shows endogenous ubiquilin, as detected by affinity-purified anti-ubiquilin-C antibody (confocal microscopy).

FIG. 6H shows overexpressed GFP-tagged ubiquilin of live HeLa cells, as seen by fluorescence microscopy which revealed accumulation of the fusion protein to the cytoplasm and to similar punctate structures.

FIG. 6I shows additional evidence for intracellular localization of ubiquilin, using a myc-tagged construct and stained with an anti-myc mAb (confocal microscopy). Bar, 25 μm.

FIGS. 7A-D show the intracellular colocalization between ubiquilin and the presenilins. HeLa cells were cotransfected with ubiquilin and either (A) wild-type PS1, (B) wild-type PS2, (C) PS2(C) deletion mutant, or (D) PS2(LC) deletion mutant and costained with appropriate goat anti-presenilin antibodies (left images) and affinity-purified rabbit anti-ubiquilin-C antibody (center images). The green (fluorescein) and red (rhodamine) confocal images in each row were merged and shown on the right, with yellow indicating colocalization of ubiquilin and presenilin proteins. Bar, 10 μm.

FIGS. 8A-D show that ubiquilin promotes increased PS2 protein accumulation. (A-D) HeLa cells, 12 h after transfection with ubiquilin (15 μg expression plasmid, lanes 1-3), PS2 (7 μg expression plasmid, lanes 4-6), or both (lanes 7-9), were either left untreated (lanes 1, 4, and 7) or treated for 5-6 h with proteasome inhibitors (20 μM synthetic lactacystin in lanes 2, 5, and 8; 40 μM MG-132 in lanes 3, 6, and 9). Equivalent amounts of protein (100 μg) from each sample were immunoblotted with (A) anti-ubiquitin, (B) anti-PS2-NH₂ terminus, (C) affinity-purified anti-ubiquilin-C, or (D) anti-tubulin antibodies. As expected, anti-ubiquitin antibodies detected larger molecular weight proteins in cells treated with proteasome inhibitors (lanes 2 and 3, 5 and 6, and 8 and 9) compared with untreated cells (lanes 1, 4, and 7). Significantly more PS2 protein (and slower migrating forms) could be seen in cells cotransfected with ubiquilin (lanes 7-9, arrowhead) compared with those transfected with PS2 alone (lanes 4-6). A doublet of weakly reactive bands was detected in all lysates, but we considered them to be nonspecific proteins. The anti-tubulin blot shows equal protein loading of each sample.

FIG. 8E shows HeLa cells that were transfected with PS2 alone (9 μg expression plasmid, lane 1) or cotransfected along with increasing amounts of ubiquilin (1, 2, 3, or 4 μg expression plasmid in lanes 2-5, respectively). Equivalent amounts of the transfected lysates were separated through an 8.5% polyacrylamide gel and immunoblotted with anti-PS2-NH₂ terminus antibody.

FIG. 8F shows the same testing procedure as in E, except with the same increasing amounts of GFP expression plasmid (lanes 2-5) instead of ubiquilin.

FIG. 8G shows the same testing procedure as in E, but proteins were separated on a 10% polyacrylamide gel and immunoblotted with anti-PS2-loop antibody. Note the absence of any detectable PS2 cleavage products corresponding to endoproteolytic PS2 cleavage in the loop. (H) The same blot shown in G or parallel blots were immunoblotted for lamin B, calreticulin, calnexin, BiP, and tubulin. The relative levels of these other endogenous proteins remained relatively unchanged compared with the PS2 levels.

FIG. 9A shows HeLa cells, electroporated with a mixture of either PS2 and GFP expression plasmids (7 μg PS2 and 15 μg pEGFP-C1) or with PS2 and ubiquilin expression plasmids (7 μg PS2 and 15 μg ubiquilin), that were pulse labeled with [³⁵S]methionine for 1 h and then chased with nonradioactive medium for 0-6 h. At appropriate time intervals (indicated above each lane), the cells were lysed and PS2 protein was immunoprecipitated using a rabbit anti-PS2-loop antibody. The immunoprecipitated proteins were separated by SDS-PAGE through an 8.5% gel, and the radioactivity of the band corresponding to full-length PS2 (arrowhead) in each lane was determined by phosphoimage analysis. The light band was likely a non-PS2 related protein whose radioactivity changed little during the chase period of the experiment and was therefore useful for normalizing protein amounts loaded in each lane.

FIG. 9B illustrates a graph showing the exponential decline of pulse-labeled PS2 protein over time. The calculated half-life of PS2 in this experiment was 3.1 and 2.9 h when coexpressed with GFP or ubiquilin, respectively. In this and in two other experiments, 1.4-1.6-fold more PS2 protein was synthesized (after normalization) when coexpressed with ubiquilin than with GFP.

FIG. 9C shows mock-electroporated HeLa cells that were pulse labeled with [³⁵S]methionine for 1 h and then chased with nonradioactive medium for 0-21 h. Ubiquilin protein was immunoprecipitated from the lysates using rabbit anti-ubiquilin-C antibodies. The radioactivity of the immunoprecipitated ubiquilin band was determined by phosphoimage analysis. This analysis revealed a small decline in radioactivity (15% reduction) over 21 h, indicating that the endogenous ubiquilin in HeLa cells is long-lived, with an estimated half-life of 90 h.

FIGS. 10A-D show anti-ubiquilin staining of human brain tissue that reveals strong staining of neurons in human brain and robust staining of NFTs and Lewy bodies of AD and PD, respectively. Sections of human brain were stained with either the preimmune or anti-ubiquilin-B and anti-ubiquilin-C antibodies. (A-D) Consecutive sections of the hippocampus of an AD afflicted brain were stained with either the (A and C) preimmune serum or with their corresponding (B) anti-ubiquilin-B and (D) anti-ubiquilin-C antibodies. (E and F). Examples of strong staining of NFTs (arrows) in hippocampal sections of AD afflicted brains with anti-ubiquilin-C antibodies. (G) Anti-ubiquilin-C antibody staining of a control nonAD human brain showing strong staining of neurons. (H) Cortical human brain section of a DLBD afflicted brain showing strong anti-ubiquilin-C staining of Lewy bodies (arrows). Bars: (A-D and G and H) 40 μm; (E and F) 20 μm.

### DETAILED DESCRIPTION OF PREFERRED EMBODIMENTS

In order to facilitate review of the various embodiments of the invention and provide an understanding of the various elements and constituents used in making and using the present invention, the following terms used in the invention description have the following meanings.

Definitions

The term “nucleic acid sequence”, as used herein, refers to an oligonucleotide, nucleotide, or polynucleotide, and fragments or portions thereof, and to DNA, cDNA or RNA of genomic or synthetic origin, which may be single- or double-stranded, and represent the sense or antisense strand.

The term “amino acid sequence”, as used herein, refers to an oligopeptide, peptide, polypeptide, or protein sequence, and fragments or portions thereof, and to naturally occurring or synthetic molecules. The term “modulate”, as used herein, refers to a change in the activity of a polypeptide. For example, modulation may cause an increase or a decrease in protein activity, binding characteristics, or any other biological, functional or immunological properties of the polypeptide.

The term “substitution”, as used herein, refers to the replacement of one or more amino acids or nucleotides by different amino acids or nucleotides, respectively. Modifications and changes can be made in the structure of a polypeptide of the present invention and still obtain a molecule having like ubiquilin peptide characteristics. For example, certain amino acids can be substituted for other amino acids in a sequence without appreciable loss of peptide activity.

In making such changes, the hydropathic index of amino acids can be considered. The importance of the hydropathic amino acid index in conferring interactive biologic function on a polypeptide is generally understood in the art (Kyte, J. and R. F. Doolittle 1982). It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within .+−0.2 is preferred, those which are within .+−0.1 are particularly preferred, and those within .+−0.5 are even more particularly preferred.

Substitution of like amino acids can also be made on the basis of hydrophilicity, particularly where the biological functional equivalent polypeptide or peptide thereby created is intended for use in immunological embodiments. U.S. Pat. No. 4,554,101, incorporated herein by reference, states that the greatest local average hydrophilicity of a polypeptide, as governed by the hydrophilicity of its adjacent amino acids, correlates with its immunogenicity and antigenicity, i.e. with a biological property of the polypeptide. As detailed in U.S. Pat. No. 4,554,101, the following hydrophilicity values have been assigned to amino acid residues: arginine (+3.0); lysine (+3.0); aspartate (+3.0.+−0.1); glutamate (+3.0.+−0.1); serine (+0.3); asparagine (+0.2); glutamine (+0.2); glycine (0); proline (−0.5.+−0.1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within .+−0.2 is preferred, those which are within .+−0.1 are particularly preferred, and those within .+−0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well known to those of skill in the art and include: arginine and lysine; glutamate and aspartate; serine and threonine; glutamine and asparagine; and valine, leucine and isoleucine (See Table 1, below). The present invention thus contemplates functional or biological equivalents of a peptide as set forth above.

TABLE 1

| Original Residue | Exemplary Substitutions |
|---|---|
| Ala | Gly; Ser |
| Arg | Lys |
| Asn | Gln; His |
| Asp | Glu |
| Cys | Ser |
| Gln | Asn |
| Glu | Asp |
| Gly | Ala |
| His | Asn; Gln |
| Ile | Leu; Val |
| Leu | Ile; Val |
| Lys | Arg |
| Met | Leu; Tyr |
| Ser | Thr |
| Thr | Ser |
| Trp | Tyr |
| Tyr | Trp; Phe |
| Val | Ile; Leu |

The term “immunologically active” refers to the capability of the natural, recombinant, or synthetic protein, or any oligopeptide thereof, to induce a specific immune response in appropriate animals or cells and to bind with specific antibodies.

The term “biologically active”, as used, refers to a protein having structural, regulatory, or biochemical functions of a naturally occurring molecule.

The term “homology”, as used herein, refers to a degree of complementarity. There may be partial homology or complete homology. A partially complementary sequence that at least partially inhibits an identical sequence from hybridizing to a target nucleic acid is referred to using the functional term “substantially homologous.” The inhibition of hybridization of the completely complementary sequence to the target sequence may be examined using a hybridization assay (Southern or northern blot, solution hybridization and the like) under conditions of high to low stringency. A substantially homologous sequence or hybridization probe will compete for and inhibit the binding of a completely homologous sequence to the target sequence under conditions of low stringency.

The term “hybridization”, as used herein, refers to any process by which a strand of nucleic acid binds with a complementary strand through base pairing. A hybridization complex may be formed in solution or between one nucleic acid sequence present in solution and another nucleic acid sequence immobilized on a solid support (e.g., paper, membranes, filters, chips, pins or glass slides, or any other approproiate substrate to which cells or their nucleic acids have been fixed).

As used herein, the term “stringent conditions” refers to conditions which permit hybridization between polynucleotide sequences and the claimed polynucleotide sequences. Suitably stringent conditions can be defined by, for example, the concentrations of salt or formamide in the prehybridization and hybridization solutions, or by the hybridization temperature, and are well known in the art. In particular, stringency can be increased by reducing the concentration of salt, increasing the concentration of formamide, or raising the hybridization temperature.

The term “specific binding”, as used herein, in reference to the interaction of an antibody and a protein or peptide, mean that the interaction is dependent upon the presence of a particular structure (i.e., the antigenic determinant or epitope) on the protein; in other words, the antibody is recognizing and binding to a specific protein structure rather than to proteins in general.

As used herein, the term “antibody” refers to intact molecules as well as fragments thereof, such as Fa, F(ab′)₂, and Fv, which are capable of binding the epitopic determinant.

The term “transformed cell”, as used herein, is a cell into which has been introduced, by means of recombinant DNA technique, a DNA molecule encoding a protein of interest.

The term “transformation”, as defined herein, describes a process by which exogenous DNA enters and changes a recipient cell. It may occur under natural or artificial conditions using various methods well known in the art. Transformation may rely on any known method for the insertion of foreign nucleic acid sequences into a prokaryotic or eukaryotic host cell. The method is selected based on the host cell being transformed and may include, but is not limited to, viral infection, electroporation, lipofection, and particle bombardment. Such “transformed” cells include stably transformed cells in which the inserted DNA is capable of replication either as an autonomously replicating plasmid or as part of the host chromosome. They also include cells which transiently express the inserted DNA or RNA for limited periods of time.

The Invention:

The present invention is based on the discovery of a novel human presenilin-interacting protein named by the present inventors as ubiquilin. Y2H interaction, GST pull-down experiments, coimmunoprecipitation studies, changes in the cellular fractionation of proteins, and colocalization of the proteins expressed in vivo provide compelling evidence that ubiquilin and the presenilins interact with one another. Ubiquilin is an important protein because it contains multiple ubiquitin-related domains typically thought to be involved in targeting proteins for degradation, yet ubiquilin promotes increased presenilin protein accumulation. Moreover, ubiquilin is highly expressed in neurons of human brain and is associated with NFTs and Lewy bodies of AD and PD brains, respectively.

Accordingly, it has been discovered that ubiquilin promotes presenilin protein accumulation and that ubiquilin facilitates increased presenilin synthesis without substantially changing presenilin protein half-life. Immunohistochemistry of human brain tissue with ubiquilin-specific antibodies revealed prominent staining of neurons. Moreover, the anti-ubiquilin antibodies robustly stained neurofibrillary tangles and Lewy bodies in AD and Parkinson's disease affected brains, respectively. The results shown herein indicate that ubiquilin may be an important modulator of presenilin protein accumulation and that ubiquilin protein is associated with neuropathological neurofibrillary tangles and Lewy body inclusions in diseased brain.

Expression Vectors

The present invention provides expression vectors comprising polynucleotides that encode ubiquilin and/or coexpression of ubiquilin and presenilin peptides. Preferably, expression vectors of the present invention comprise polynucleotides that encode polypeptides comprising the amino acid residue sequence of SEQ ID NOs: 2 or 4 (amino acid sequences of ubiquilin 595 and 589, respectively) and SEQ ID NO. 5 or 6 (amino acid sequences of Presenilin 1 and 2, respectively). More preferably, expression vectors of the invention comprise polynucleotides operatively linked to an enhancer-promoter. More preferably still, expression vectors of the invention comprise a polynucleotide operatively linked to a prokaryotic or eukaryotic promoter.

A promoter is a region of a DNA molecule typically within about 100 nucleotide pairs in front of (upstream of) the point at which transcription begins (i.e., a transcription start site). That region typically contains several types of DNA sequence elements that are located in similar relative positions in different genes. As used herein, the term “promoter” includes what is referred to in the art as an upstream promoter region, a promoter region or a promoter of a generalized eukaryotic RNA Polymerase II transcription unit.

Another type of discrete transcription regulatory sequence element is an enhancer. An enhancer provides specificity of time, location and expression level for a particular encoding region (e.g., gene). A major function of an enhancer is to increase the level of transcription of a coding sequence in a cell that contains one or more transcription factors that bind to that enhancer. Unlike a promoter, an enhancer can function when located at variable distances from transcription start sites so long as a promoter is present.

As used herein, the phrase “enhancer-promote” means a composite unit that contains both enhancer and promoter elements. An enhancer-promoter is operatively linked to a coding sequence that encodes at least one gene product. As used herein, the phrase “operatively linked” means that an enhancer-promoter is connected to a coding sequence in such a way that the transcription of that coding sequence is controlled and regulated by that enhancer-promoter. Means for operatively linking an enhancer-promoter to a coding sequence are well known in the art. As is also well known in the art, the precise orientation and location relative to a coding sequence whose transcription is controlled, is dependent inter alia upon the specific nature of the enhancer-promoter. Thus, a TATA box minimal promoter is typically located from about 25 to about 30 base pairs upstream of a transcription initiation site and an upstream promoter element is typically located from about 100 to about 200 base pairs upstream of a transcription initiation site. In contrast, an enhancer can be located downstream from the initiation site and can be at a considerable distance from that site.

An enhancer-promoter used in a vector construct of the present invention can be any enhancer-promoter that drives expression in a cell to be transfected. By employing an enhancer-promoter with well-known properties, the level and pattern of gene product expression can be optimized. A coding sequence of an expression vector is operatively linked to a transcription terminating region. RNA polymerase transcribes an encoding DNA sequence through a site where polyadenylation occurs. Typically, DNA sequences located a few hundred base pairs downstream of the polyadenylation site serve to terminate transcription. Those DNA sequences are referred to herein as transcription-termination regions. Those regions are required for efficient polyadenylation of transcribed messenger RNA (RNA). Transcription-terminating regions are well known in the art. A preferred transcription-terninating region is derived from a bovine growth hormone gene.

Exemplary vectors include the mammalian expression vectors of the pCMV family including pCMV6b and pCMV6c (Chiron Corp., Emeryville Calif.) and pRc/CMV (invitrogen, San Diego, Calif). In certain cases, and specifically in the case of these individual mammalian expression vectors, the resulting constructs can require co-transfection with a vector containing a selectable marker such as pSV2neo. Via co-transfection into a dihydrofolate reductase-deficient Chinese hamster ovary cell line, such as DG44, clones expressing the presently claimed peptides by virtue of DNA incorporated into such expression vectors can be detected.

A DNA molecule of the present invention can be incorporated into a vector using a number of techniques which are well known in the art. For instance, the vector pUC 18 has been demonstrated to be of particular value. Likewise, the related vectors M13 mp 18 and M13 mp19 can be used in certain embodiments of the invention, in particular, in performing dideoxy sequencing.

Where expression of recombinant polypeptides of the present invention is desired and a eukaryotic host is contemplated, it is most desirable to employ a vector, such as a plasmid, that incorporates a eukaryotic origin of replication. Additionally, for the purposes of expression in eukaryotic systems, one desires to position the peptide encoding sequence adjacent to and under the control of an effective eukaryotic promoter such as promoters used in combination with Chinese hamster ovary cells. To bring a coding sequence under control of a promoter, whether it is eukaryotic or prokaryotic, what is generally needed is to position the 5′ end of the translation initiation side of the proper translational reading frame of the polypeptide between about 1 and about 50 nucleotides 3′ of or downstream with respect to the promoter chosen.

The pRc/CMV vector (available from hivitrogen) is an exemplary vector for expressing a peptide of the present invention in mammalian cells, particularly COS and CHO cells. A polypeptide of the present invention under the control of a CMV promoter can be efficiently expressed in mammalian cells. The pCMV plasmids are a series of mammalian expression vectors of particular utility in the present invention. The vectors are designed for use in essentially all cultured cells and work extremely well in SV40-transformed simian COS cell lines. The pCMV1, 2, 3, and 5 vectors differ from each other in certain unique restriction sites in the polylinker region of each plasmid. The pCMV4 vector differs from these 4 plasmids in containing a translation enhancer in the sequence prior to the polylinker. While they are not directly derived from the pCMV1-5 series of vectors, the functionally similar pCMV6b and c vectors are available from the Chiron Corp. of Emeryville, Calif. and are identical except for the orientation of the polylinker region which is reversed in one relative to the other.

The universal components of the pCMV plasmids are as follows. The vector backbone is pTZ18R (Pharmacia), and contains a bacteriophage f1 origin of replication for production of single stranded DNA and an ampicillin-resistance gene. The CMV region consists of nucleotides −760 to +3 of the powerful promoter-regulatory region of the human cytomegalovirus (Towne stain) major immediate early gene (Thonsen, et al., 1984; Boshart, et al., 1985). The human growth hormone fragment (hGH) contains transcription termination and poly-adenylation signals representing sequences 1533 to 2157 of this gene (Seeburg, 1982). There is an Alu middle repetitive DNA sequence in this fragment. Finally, the SV40 origin of replication and early region promoter-enhancer derived from the pcD-X plasmid (HindIi to PstI fragment) described in Okayama, et al., (1983). The promoter in this fragment is oriented such that transcription proceeds away from the CMV/hGH expression cassette.

The pCMV plasmids are distinguishable from each other by differences in the polylinker region and by the presence or absence of the translation enhancer. The stating pCMV1 plasmid has been progressively modified to render an increasing number of unique restriction sites in the polylinker region. To create pCMV2, one of two EcoRI sites in pCMV1 were destroyed. To create pCMV3, pCMV1 was modified by deleting a short segment from the SV40 region (Stul to EcoRI), and in so doing made unique the PstI, SalI, and BamnHI sites in the polylinker. To create pCMV4, a synthetic fragment of DNA corresponding to the 5′-untranslated region of a mRNA transcribed from the CMV promoter was added C. To create pCMV5, a segment of DNA (HpaI to EcoRI) was deleted from the SV40 origin region of pCMV1 to render unique all sites in the starting polylinker. The pCMV vectors have been successfully expressed in simian COS cells, mouse L cells, CHO cells, and HeLa cells.

Transfected Cells

In yet another embodiment, the present invention provides recombinant host cells transformed or transfected with a polynucleotide that includes nucleotide sequences SEQ ID NOs. 1 and 3 (nucleotide sequences encoding for ubiquilin 595 and ubiquilin 589, respectively) and SEQ ID NOs. 7 and 8 (nucleotide sequences for presenilin 1 and 2, respectively. Preferably the nucleotide sequence encodes for encodes a ubiquilin and/or ubiquilin-presenilin peptide.

Preferably, recombinant host cells of the present invention are transfected with a polynucleotide that encodes for a peptide having an amino acid residue sequence selected from SEQ ID NOs 2 or 4 and SEQ ID NOs: 5 or 6. Means of transforming or transfecting cells with exogenous polynucleotide such as DNA molecules are well known in the art and include techniques such as calcium-phosphate- or DEAE-dextran-mediated transfection, protoplast fusion, electroporation, liposome mediated transfection, direct microinjection and adenovirus infection.

The most widely used method is transfection mediated by either calcium phosphate or DEAE-dextran. Although the mechanism remains obscure, it is believed that the transfected DNA enters the cytoplasm of the cell by endocytosis and is transported to the nucleus. Depending on the cell type, up to 90% of a population of cultured cells can be transfected at any one time. Because of its high efficiency, transfection mediated by calcium phosphate or DEAE-dextran is the method of choice for experiments that require transient expression of the foreign DNA in large numbers of cells. Calcium phosphate-meddiated transfection is also used to establish cell lines that integrate copies of the foreign DNA, which arc usually arranged in head-to-tail tandem arrays into the host cell genome.

In the protoplast fusion method, protoplasts derived from bacteria carrying high numbers of copies of a plasmid of interest are mixed directly with cultured mammalian cells. After fusion of the cell membranes (usually with polyethylene glycol), the contents of the bacteria are delivered into the cytoplasm of the mammalian cells and the plasmid DNA is transported to the nucleus. Protoplast fusion is not as efficient as transfection for many of the cell lines that are commonly used for transient expression assays, but it is useful for cell lines in which endocytosis of DNA occurs inefficiently. Protoplast fusion frequently yields multiple copies of the plasmid DNA tandemly integrated into the host chromosome.

The application of brief, high-voltage electric pulses to a variety of mammalian and plant cells leads to the formation of nanometer-sized pores in the plasma membrane. DNA is taken directly into the cell cytoplasm either through these pores or as a consequence of the redistribution of membrane components that accompanies closure of the pores. Electroporation can be extremely efficient and can be used both for transient expression of cloned genes and for establishment of cell lines that carry integrated copies of the gene of interest. Electroporation, in contrast to calcium phosphate-mediated transfection and protoplast fusion, frequently gives rise to cell lines that carry one, or at most a few, integrated copies of the foreign DNA.

Liposome transfection involves encapsulation of DNA and RNA within liposomes, followed by fusion of the liposomes with the cell membrane. The mechanism of how DNA is delivered into the cell is unclear but transfection efficiencies can be as high as 90%.

Direct microinjection of a DNA molecule into nuclei has the advantage of not exposing DNA to cellular compartments such as low-pH endosomes. Microinjection is therefore used primarily as a method to establish lines of cells that carry integrated copies of the DNA of interest.

A transfected cell can be prokaryotic or eukaryotic. Preferably, the host cells of the invention are eukaryotic host cells.

In another aspect, the recombinant host cells of the present invention are prokaryotic host cells, such as bacterial cells. In general, prokaryotes are preferred for the initial cloning of DNA sequences and constructing the vectors useful in the invention. For example, E. coli K12 strains can be particularly useful. Other microbial strains which can be used include E. coli B, and E. coli X1776 (ATCC No. 31537). These examples are, of course, intended to be illustrative rather than limiting.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli can be transformed using pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, promoters which can be used by the microbial organism for expression of its own polypeptides.

Those promoters most commonly used in recombinant DNA construction include the beta-lactamase (penicillinase) and lactose promoter systems and a tryptophan (TRP) promoter system. While these are the most commonly used, other microbial promoters have been discovered and utilized, and details concerning their nucleotide sequences have been published, enabling a skilled worker to introduce functional promoters into plasmid vectors.

In addition to prokaryotes, eukaryotic microbes, such as yeast can also be used. Saccharomyces cerevisiae or common baker's yeast is the most commonly used among eukaryotic microorganisms, although a number of other strains are commonly available. For expression in Saccharomyces, the plasmid YRp7, for example, is commonly used. This plasmid already contains the trp1 gene which provides a selection marker for a mutant strain of yeast lacking the ability to grow in tryptophan, for example ATCC No. 44076 or PEP4-1. The presence of the trp1 lesion as a characteristic of the yeast host cell genome then provides an effective environment for detecting transformation by growth in the absence of tryptophan. Suitable promoter sequences in yeast vectors include the promoters for 3-phosphoglycerate kinase or other glycolytic enzymes such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. In constructing suitable expression plasmids, the termination sequences associated with these genes are also introduced into the expression vector downstream from the sequences to be expressed to provide polyadenylation of the mRNA and termination. Other promoters, which have the additional advantage of transcription controlled by growth conditions are the promoter region for alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization. Any plasmid vector containing a yeast-compatible promoter, origin or replication and termination sequences is suitable.

In addition to microorganisms, cultures of cells derived from multicellular organisms can also be used as hosts. In principle, any such cell culture is workable, whether from vertebrate or invertebrate culture. However, interest has been greatest in vertebrate cells, and propagation of vertebrate cells in culture (tissue culture) has become a routine procedure in recent years. Examples of such useful host cell lines are AtT-20, VERO and HeLa cells, Chinese hamster ovary (CHO) cell lines, and W138, BHK, COSM6, COS-1, COS-7, 293 and MDCK cell lines. Expression vectors for such cells ordinarily include (if necessary) an origin of replication, a promoter located upstream of the gene to be expressed, along with any necessary ribosome binding sites, RNA splice sites, polyadenylation site, and transcriptional terminator sequences.

For use in mammalian cells, the control functions on the expression vectors are often derived from viral material. For example, commonly used promoters are derived from polyoma, Adenovirus 2, Cytomegalovirus and most frequently Simian Virus 40 (SV40). The early and late promoters of SV40 virus are particularly useful because both are obtained easily from the virus as a fragment which also contains the SV40 viral origin of replication. Smaller or larger SV40 fragments can also be used, provided there is included the approximately 250 bp sequence extending from the HindIII site toward the BglI site located in the viral origin of replication. Further, it is also possible, and often desirable, to utilize promoter or control sequences normally associated with the desired gene sequence, provided such control sequences are compatible with the host cell systems.

Preparing a Ubiquilin Peptide or Transcription

The present invention contemplates a process of preparing a ubiquilin or a ubiquilin-presenilin peptide comprising transfecting cells with a polynucleotide that encodes the preferred peptides to produce transformed host cells; and maintaining the transformed host cells under biological conditions sufficient for expression of the polypeptide. Preferably, the transformed host cells are eukaryotic cells.

A host cell used in the process is capable of expressing a functional, recombinant ubiqluilin or ubiquilin-presenilin peptides. Following transfection, the cell is maintained under culture conditions for a period of time sufficient for expression of the preferred peptide. A suitable time depends inter alia upon the cell type used and is readily determinable by a skilled artisan. Typically, maintenance time is from about 2 to about 14 days. Culture conditions are well known in the art and include ionic composition and concentration, temperature, pH and the like. Typically, transfected cells are maintained under culture conditions in a culture medium. Suitable medium for various cell types are well known in the art. In a preferred embodiment, temperature is from about 20° C. to about 50° C. pH is preferably from about a value of 6.0 to a value of about 8.0, more preferably from about a value of about 6.8 to a value of about 7.8 and, most preferably about 7.4. Other biological conditions needed for transfection and expression of an encoded protein are well known in the art.

A recombinant ubiquilin peptide or ubiquilin-presenilin polypeptide is recovered or collected either from the transfected cells or the medium in which those cells are cultured. Recovery comprises isolating and purifying the recombinant polypeptide. Isolation and purification techniques for polypeptides are well known in the art and include such procedures as precipitation, filtration, chromatography, electrophoresis and the like.

Antibodies

In still another embodiment, the present invention provides antibodies immunoreactive with a polypeptide of the present invention. Preferably, the antibodies of the invention are monoclonal antibodies. Means for preparing and characterizing antibodies are well known in the art.

Briefly, a polyclonal antibody is prepared by immunizing an animal with an immunogen comprising a polypeptide or polynucleotide of the present invention, and collecting antisera from that immunized animal. A wide range of animal species can be used for the production of antisera. Typically, the animal used for production of anti-antisera is a rabbit, a mouse, a rat, a hamster or a guinea pig. Because of the relatively large blood volume of rabbits, a rabbit is a preferred choice for production of polyclonal antibodies. As is well known in the art, a given polypeptide or polynucleotide may vary in its immunogenicity. It is often necessary therefore to couple the immunogen (e.g., a polypeptide or polynucleotide of the present invention) with a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide or a polynucleotide to a carrier protein are well known in the art and include glutaraldehyde, M maleimidobenzoyl-N-hydroxysuccinimide ester, carbodiilmide and bis-biazotized benzidine.

As is also well known in the art, immunogenicity to a particular immunogen can be enhanced by the use of non-specific stimulators of the immune response known as adjuvants. Exemplary and preferred adjuvants include complete Freund's adjuvant, incomplete Freund's adjuvants and aluminum hydroxide adjuvant.

The amount of immunogen used of the production of polyclonal antibodies varies inter alia, upon the nature of the immunogen as well as the animal used for immunization. A variety of routes can be used to administer the immunogen (subcutaneous, intramuscular, intradermal, intravenous and intraperitoneal. The production of polyclonal antibodies is monitored by sampling blood of the immunized animal at various points following immunization. When a desired level of immunogenicity is obtained, the immunized animal can be bled and the serum isolated and stored.

In another aspect, the present invention contemplates a process of producing an antibody immunoreactive with the ubiquilin peptide comprising the steps of (a) transfecting recombinant host cells with polynucleotide that encodes for the ubiquilin peptide; (b) culturing the host cells undcr conditions sufficient for expression of the peptide; (c) recovering the peptide; and (d) preparing the antibodies to the polypeptide.

Typically, a monoclonal antibody of the present invention can be readily prepared by a technique which involves first immunizing a suitable animal with a selected antigen (e.g., a polypeptide or polynucleotide of the present invention) in a manner sufficient to provide an immune response. Rodents such as mice and rats are preferred animals. Spleen cells from the immunized animal are then fused with cells of an immortal myeloma cell. Where the immunized animal is a mouse, a preferred myeloma cell is a murine NS-1 myeloma cell.

The fused spleen/myeloma cells are cultured in a selective medium to select fused spleen/myeloma cells from the parental cells. Fused cells are separated from the mixture of non-fused parental cells, for example, by the addition of agents that block the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides. Where azaserine is used, the media is supplemented with hypoxanthine. This culturing provides a population of hybridomas from which specific hybridomas are selected. Typically, selection of hybridomas is performed by culturing the cells by single-clone dilution in microliter plates, followed by testing the individual clonal supernatants for reactivity with an antigen-polypeptide. The selected clones can then be propagated indefinitely to provide the monoclonal antibody.

By way of specific example, to produce an antibody of the present invention, mice are injected intraperitoneally with between about 1-200 μg of an antigen comprising a polypeptide of the present invention. B lymphocyte cells are stimulated to grow by injecting the antigen in association with an adjuvant such as complete Freund's adjuvant (a non-specific stimulator of the immune response containing killed Mycobacterium tuberculosis). At some time (e.g., at least two weeks) after the first injection, mice are boosted by injection with a second dose of the antigen mixed with incomplete Freund's adjuvant. A few weeks after the second injection, mice are tail bled and the sera titered by immunoprecipitation against radiolabeled antigen. Preferably, the process of boosting and titering is repeated until a suitable titer is achieved. The spleen of the mouse with the highest titer is removed and the spleen lymphocytes are obtained by homogenizing the spleen with a syringe. Typically, a spleen from an immunized mouse contains approximately 5×10⁷ to 2×10⁸ lymphocytes.

Mutant lymphocyte cells known as myeloma cells are obtained from laboratory animals in which such cells have been induced to grow by a variety of well-known methods. Myeloma cells lack the salvage pathway of nucleotide biosynthesis. Because myeloma cells are tumor cells, they can be propagated indefinitely in tissue culture, and are thus denominated immortal. Numerous cultured cell lines of myeloma cells from mice and rats, such as murine NS-1 myeloma cells, have been established.

Myeloma cells are combined under conditions appropriate to foster fusion with the normal antibody-producing cells from the spleen of the mouse or rat injected with the antigen/polypeptide of the present invention. Fusion conditions include, for example, the presence of polyethylene glycol. The resulting fused cells are hybridoma cells. Like myeloma cells, hybridoma cells grow indefinitely in culture. Hybridoma cells are separated from unfused myeloma cells by culturing in a selection medium such as HAT media (hypoxanthine, aminopterin, thymidine). Unfused myeloma cells lack the enzymes necessary to synthesize nucleotides from the salvage pathway because they are killed in the presence of aminopterin, methotrexate, or azaserine. Unfused lymphocytes also do not continue to grow in tissue culture. Thus, only cells that have successfully fused (hybridoma cells) can grow in the selection media. Each of the surviving hybridoma cells produces a single antibody. These cells are then screened for the production of the specific antibody immunoreactive with an antigen/polypeptide of the present invention. Single cell hybridomas are isolated by limiting dilutions of the hybridomas. The hybridomas are serially diluted many times and, after the dilutions are allowed to grow, the supernatant is tested for the presence of the monoclonal antibody. The clones producing that antibody are then cultured in large amounts to produce an antibody of the present invention in convenient quantity.

By use of a monoclonal antibody of the present invention, specific polypeptides and polynucleotide of the invention can be recognized as antigens, and thus identified. Once identified, those polypeptides and polynucleotide can be isolated and purified by techniques such as antibody-affinity chromatography. In antibody-affinity chromatography, a monoclonal antibody is bound to a solid substrate and exposed to a solution containing the desired antigen. The antigen is removed from the solution through an immunospecific reaction with the bound antibody. The polypeptide or polynucleotide is then easily removed from the substrate and purified.

A Process of Detecting the Polypeptides of the Present Invention

Alternatively, the present invention provides a process of detecting a polypeptide of the present invention, wherein the process comprises immunoreacting the polypeptide with antibodies prepared according to a process described above to form an antibody-polypeptide conjugate and detecting the conjugates.

The present invention provides a process of screening a biological sample for the presence of a ubiquilin peptide or a ubiquilin-presenilin polypeptide. A biological sample to be screened can be a biological fluid such as extracellular or intracellular fluid or a cell or tissue extract or homogenate. A biological sample can also be an isolated cell (e.g., in culture) or a collection of cells such as in a tissue sample or histology sample. A tissue sample can be suspended in a liquid medium or fixed onto a solid support such as a microscope slide.

In accordance with a screening assay process, a biological sample is exposed to an antibody immunoreactive with the ubiquilin peptide whose presence is being assayed. Typically, exposure is accomplished by forming an admixture in a liquid medium that contains both the antibody and the candidate peptide. Either the antibody or the sample with the peptide can be affixed to a solid support (e.g., a column or a microliter plate). The biological sample is exposed to the antibody under biological reaction conditions and for a period of time sufficient for antibody-polypeptide conjugate formation. Biological reaction conditions include ionic composition and concentration, temperature, pH and the like. Ionic composition and concentration can range from that of distilled water to a 2 molal solution of NaCl. Temperature preferably is from about 25° C. to about 40° C. pH is preferably from about a value of 4.0 to a value of about 9.0, more preferably from about a value of 6.5 to a value of about 8.5 and, even more preferably from about a value of 7.0 to a value of about 7.5. The only limit on biological reaction conditions is that the conditions selected allow for antibody-polypeptide conjugate formation and that the conditions do not adversely affect either the antibody or the peptide.

Exposure time will vary inter alia with the biological conditions used, the concentration of antibody and peptide and the nature of the sample (e.g., fluid or tissue sample). Means for determining exposure time are well known to one of ordinary skill in the art. Typically, where the sample is fluid and the concentration of peptide in that sample is about 10⁻¹⁰ M, exposure time is from about 10 minutes to about 200 minutes.

The presence of a ubiquilin peptide in the sample is detected by detecting the formation and presence of antibody-peptide conjugates. Means for detecting such antibody-antigen (e.g., peptide) conjugates or complexes are well known in the art and include such procedures as centrifugation, affinity chromatography and the like, binding of a secondary antibody to the antibody-candidate peptide complex.

In one embodiment, detection is accomplished by detecting an indicator affixed to the antibody. Exemplary and well known such indicators include radioactive labels (e.g., ³²P, 125 I, ¹⁴C), a second antibody or an enzyme such as horse radish peroxidase. Means for affixing indicators to antibodies are well known in the art. Commercial kits are available.

Pharmaceutical Compositions

In a preferred embodiment, the present invention provides pharmaceutical compositions comprising ubiquilin or a combination of ubiquilin and another protein, such as a presenilin and a physiologically acceptable carrier. More preferably, a pharmaceutical composition comprises ubiquilin having the amino acid residue sequence of SEQ ID NO:2 or SEQ ID NO: 4. Additionally, a pharmaceutical composition of the invention comprises a polynucleotide that encodes ubiquilin and a physiologically acceptable carrier.

A composition of the present invention is typically administered parenterally in dosage unit formulations containing standard, well-known nontoxic physiologically acceptable carriers, adjuvants, and vehicles as desired. The term parenteral as used herein includes intravenous, intramuscular, intraarterial injection, or infusion techniques.

Injectable preparations, for example sterile injectable aqueous or oleaginous suspensions, are formulated according to the known art using suitable dispersing or wetting agents and suspending agents. The sterile injectable preparation can also be a sterile injectable solution or suspension in a nontoxic parenterally acceptable diluent or solvent, for example, as a solution in 1,3-butanediol.

Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid find use in the preparation of injectables.

Preferred carriers include neutral saline solutions buffered with phosphate, lactate, Tris, and the like. Of course, one purifies the vector sufficiently to render it essentially free of undesirable contaminants, such as defective interfering adenovirus particles or endotoxins and other pyrogens such that it does not cause any untoward reactions in the individual receiving the vector construct. A preferred means of purifying the vector involves the use of buoyant density gradients, such as cesium chloride gradient centrifugation.

A transfected cell can also serve as a carrier. By way of example, a liver cell can be removed from an organism, transfected with a polynucleotide of the present invention using methods set forth above and then the transfected cell returned to the organism (e.g. injected intravascularly).

Assay Kits

In another aspect, the present invention contemplates diagnostic assay kits for detecting the presence of antibodies specific for ubiquilin of the present invention in biological samples, where the kits comprise a first container containing a ubiquilin peptide capable of immunoreacting with antibodies in biological samples, in an amount sufficient to perform at least one assay. Preferably, assay kits of the invention further comprise a second container containing a second antibody that immunoreacts with the first antibody. Preferably the antibodies used in the assay kits of the present invention are monoclonal antibodies. Even more preferably, the peptides are affixed to a solid support. More preferably still, the first and second antibodies comprise an indicator, and, preferably, the indicator is a radioactive label or an enzyme. The reagents of the kit can be provided as a liquid solution, attached to a solid support or as a dried powder. Preferably, when the reagent is provided in a liquid solution, the liquid solution is an aqueous solution. Preferably, when the reagent provided is attached to a solid support, the solid support can be chromatograph media or a microscope slide.

### EXPERIMENTAL PROCEDURES

Yeast Two-Hybrid Library Screening

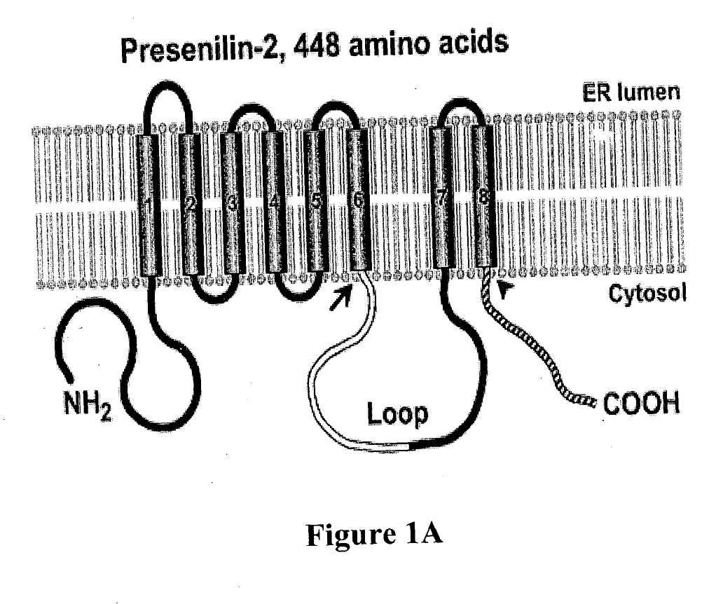

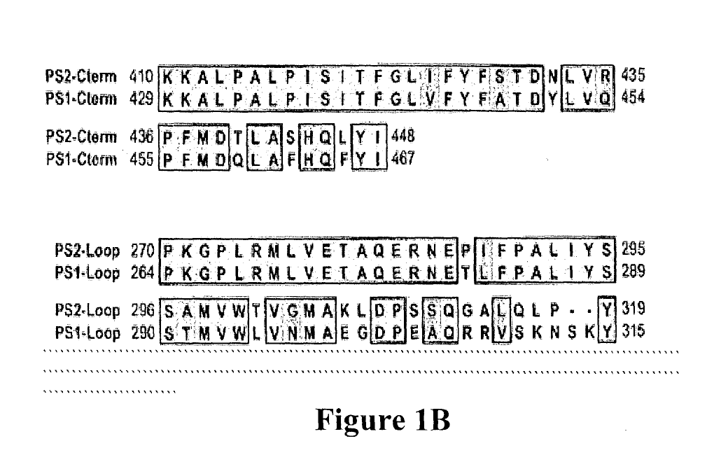

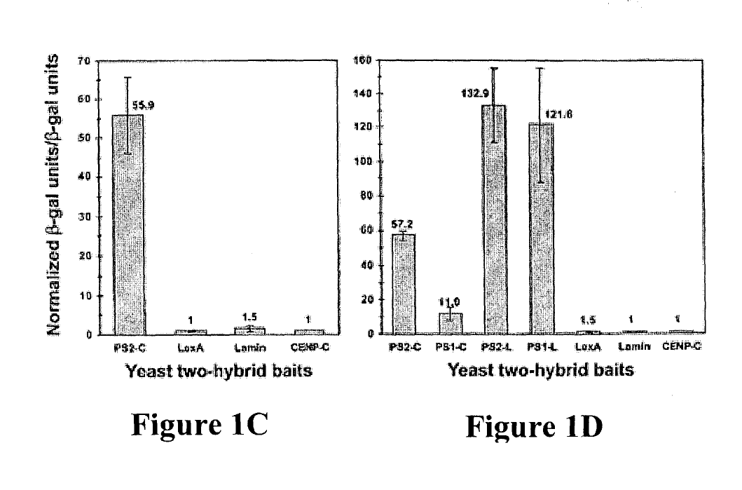

The yeast two-hybrid (Y2H) procedure that utilizes the LexA/transactivation system was performed, as described previously (Stabler, et al. 1999). The PS2 COOH-terminal 39-amino acids (residues K410-I1448) (see FIG. 1a and FIG. b) were used as bait to screen a human fetal brain cDNA library. Out of 10⁷ primary yeast transformants, 50 clones were selected for further analysis based on growth in the absence of leucine and a substantial blue color change when grown on 5-bromo-4-chloro-3-indolyl-β-D-galactopyranoside (X-gal) plates. False positives were subsequently eliminated based on their interactions with negative-control baits, such as LexA alone, nuclear lamin B, and CENP-C centromere protein. Plasmid DNAs were isolated from clones that fulfilled the above requirements, and the DNA sequences of their inserts were determined and compared with the GenBank/EMBL/DDBJ databases using the BLAST search program. Two of the interactors contained overlapping portions of the 3′ end of a novel human transcript, (which was named ubiquilin for a protein with ubiquitin-related protein domains that interacts with the presenilins), were identified in a yeast two-hybrid screen (Y2H) for proteins that interact with the PS2 COOH-terminal sequence (FIG. 1a and FIG. b). Interaction of ubiquilin proteins with various PS1 and PS2 baits were quantified in liquid culture assays by the enzymatic conversion of o-nitrophenyl-β-D-galactopyranoside (ONPG) to o-nitrophenol and D-galactose by β-galactosidase enzyme encoded by an Y2H reporter plasmid.

Rapid Amplifilcation of cDNA Ends (RACE)

To clone the entire ubiquilin ORF, 5′RACE was performed. 5′RACE, using strategically designed ubiquilin-specific primers, was performed to obtain the full-length coding sequence of ubiquilin. The PCR reactions were carried out according to the instructions provided by the manufacturer, using an AP1 adaptor-ligated human adult brain cDNA library as template and the Advantage cDNA Polymerase Mix (CLONTECH Laboratories, Inc.). This yielded 1,053 bps of the 5′ additional upstream sequence, including an in-frame methionine codon located at the beginning of an ubiquitin-like (UB) domain. The full-length ubiquilin open reading frame (ORF) was subsequently obtained by sequencing a human expressed sequence tag (EST) clone (American Type Culture Collection), which provided an extra 108 bps of 5′ sequence, including the ATG start codon, a Kozak consensus sequence, and an upstream in-frame stop codon. In addition, comparison of the sequence with the TIGR Tentative Human Consensus database returned another independent EST sequence (THC296552), which corroborated the ATG start codon.

Conceptual translation from this methionine predicted a 559-amino acid protein. However, this methionine was unlikely to be the authentic start codon since translation from it produced a protein that was noticeably smaller, as seen by SDS-PAGE, than endogenous ubiquilin detected using pAb raised to the COOH-terminal 218 residues of ubiquilin (data not shown). Subsequent EST database searches and sequencing of putative overlapping clones yielded a clone with a longer 5′ end, which included an upstream in-frame methionine codon (corresponding to a 595-amino acid protein). Several lines of evidence indicate that this methionine codon was the authentic start methionine of ubiquilin. (a) This start methionine contained a Kozak consensus sequence and an upstream in-frame stop codon. (b) Polypeptides translated from this start codon matched the endogenous ubiquilin protein in size, which was 66 kD in HeLa and other human cell types. (c) Mammalian overexpression of the complete ubiquilin ORF (see FIG. 3, D-F), in vitro transcribed and translated ubiquilin in rabbit reticulocyte lysates (see FIG. 3G), and human ubiquilin protein synthesized in bacteria (see FIG. 3H) all generated 66-kD proteins. These data indicated that the complete ubiquilin ORF had been cloned and that the protein was probably not extensively modified in mammalian cells, since bacterially and eukaryotically expressed human ubiquilin proteins have similar molecular masses when separated by SDS-PAGE.

Interaction between ubiquilin and the presenilins was quantified in Y2H β-galactosidase liquid culture assays. A partial ubiquilin clone (encoding the COOH-terminal 218 residues), isolated from the Y2H screen, and a near full-length clone (encoding residues 37-595) both bound to the PS2-COOH-terrninal bait >55-fold compared with negative-control baits (FIG. 1C and FIG. D, respectively). However, the longer ubiquilin clone interacted less strongly with the PS1-COOH-terminal bait. This interaction nevertheless was still >12-fold compared with the negative-control baits (FIG. 1D). It was also tested whether ubiquilin would interact with portions of the loop regions of the presenilins (FIGS. 1A and 1B) that were used in another Y2H study (Stabler, et al. 1999). Interestingly, the near full-length ubiquilin construct also interacted well with both PS2 and PS1 loop sequences, and, surprisingly, the interaction was approximately twice as strong as with the PS2-COOH-terminal bait (FIG. 1D). This suggested that two separate regions within the presenilin proteins could interact with ubiquilin.

Northern Blot Analysis

cDNA hybridization probes were ³²P-radiolabeled by random primer labeling of 100 ng of two ubiquilin cDNA restriction fragments, X (bases 1,132-1,860) and Y (bases 284-967), shown in FIG. 2 II. Each probe was hybridized to a different human mRNA Northern blot, Human Multiple Tissue Northern Blot I, and Human Brain MTN II, respectively (CLONTECH Laboratories, Inc.). After hybridization, the blots were washed twice with 2.0×SSC and 0.05% SDS at 50° C. and then twice again with 0.1×SSC and 0.1% SDS at 50° C., beforc exposure to autoradiography film. Both blots were subsequently stripped in near-boiling temperature 0.5% SDS and then reprobed with radiolabeled human β-actin cDNA control probe (CLONTECH Laboratories, Inc.). The band intensities were quantified using GelExpert 97 2.0 (Nucleotech Corp.), divided by the β-actin control band intensity from the same lane, and then normalized against the lowest value.

Northern blot analysis of human tissues indicated that ubiquilin mRNA is widely expressed as a major 4.4-kb transcript, with several smaller differentially expressed minor transcripts (FIG. 2 II, X; FIG. 3A). Comparison of ubiquilin band intensities after correction for mRNA loading (β-actin control bands) and normalized to the skeletal muscle value suggested that expression of the 4.4-kb transcript was highest in brain and pancreas (FIG. 3C). Additional Northern blot analysis of mRNA from different subregions of the human brain revealed slight variation (1.0-1.5-fold) of ubiquilin expression with the same 4.4-kb transcript as the predominant species (FIG. 2 II, Y; FIG. 3B and FIG. 3C).

The chromosomal location of the ubiquilin gene using PCR radiation hybrid mapping was determined (see Materials and Methods; data not shown). This procedure resulted in an unambiguous assignment of ubiquilin nearby to two loci, CHLC.GATA22H04 and CHLC.GATA81C04, located on chromosome 9q22 close to the 9q21.3 border. Interestingly, the chromosome region to which ubiquilin maps is thought to contain a susceptibility gene(s) involved in late-onset AD (Kehoe, et al. 1999).

The complete ubiquilin ORF consists of 595 residues, with a sequence rich in glutamines and serines, 10.9% and 11.1%, respectively, but lacking any cysteines (FIG. 4). The protein has a calculated isoelectric point (pI) of 4.97 and is predicted to be soluble, based on having a high hydrophilic profile using MacVector 6.5 software (Oxford Molecular Group; data not shown). Currently known protein structural motifs residing within ubiquilin include an NH₂-terminal UB domain and a COOH-terminal ubiquitin- associated (UBA) domain, both of which have been implicated in targeting and degradation of proteins by the ubiquitin-proteasome pathway. The predicted ubiquilin protein also contains numerous regularly spaced asparagine-proline (Asn-Pro) repeats of unknown function (FIG. 4). When first sequenced, no apparent mammalian homologue for ubiquilin had been deposited in GenBank/EMBL/DDBJ databases. However, there was significant homology to several full-length genes of unknown function: the C. elegans F15C11.2 gene (36% identical) and two related genes in Arabidopsis thaliana (34 and 35% identical). In addition, the protein had weak homology to the Saccharomyces cerevisiae DSK2 gene (31% identical), which can suppress a KAR1 defect (Biggins, et al. 1996). KAR1 is an essential gene involved in the duplication of the yeast microtubule-organizing center known as the spindle pole body. Interestingly, ubiquilin homology to DSK2 was primarily confined to the UB and UBA domains.

Bacterial Expression of Ubiquilin Proteins

To express full-length nonfusion ubiquilin protein in bacteria, an NcoI/XhoI cDNA fragment containing the entire ubiquilin ORF was subcloned into pET- 15b (Novagen) and then transformed into Escherichia coli BL21(DE3). Ubiquilin protein expression was induced with 1.0 mM IPTG at 37° C. for 2-3 h. Bacteria were lysed, and the protein extracts were separated by SDS-PAGE. Proteins were transferred onto nitrocellulose filters and immunoblotted with anti-ubiquilin pAb. In addition, various partial-length GST-ubiquilin fusion proteins were constructed, expressed, and purified using the GST-protein purification procedures described previously (Janicki and Monteiro 1997). The specific subregions of ubiquilin that were expressed are depicted in FIG. 2 III.

GST Pull-Down Binding Assay

Various purified GST-ubiquilin fusion proteins already bound to glutathione-agarose beads were incubated with 2.5 μl [³⁵S]methionine-radiolabeled presenilin protein, in addition to protease inhibitors and 0.8% BSA at 4° C. for 1 h with rocking. The beads were washed several times with pull-down buffer (0.5% NP-40 in 1.0×PBS), once with 200 mM KC1 in pull-down buffer, and several more times with pull-down buffer alone. Supernatants and washes were discarded, whereas the bound proteins on the beads were mixed with sample loading buffer (Janicki and Monteiro 1999) and separated by SDS-PAGE. Then, the gel was stained with Coomassie blue to ensure equal loading of GST-ubiquilin fusion proteins. The gels were dried and exposed to autoradiography film.

GST-fusion proteins containing the UBA domain and nine COOH-terminal residues (QHHSSISVS) of ubiquilin were necessary and sufficient to bind [³⁵S]methionine-radiolabeled PS2 and PS1 in a GST pull-down assay (FIG. 5A and FIG. B, respectively). Interestingly, slower migrating forms of both presenilins were much more tightly bound than the full-length forms. Whether the slower migrating forms are more ubiquitinated, extensively modified, or merely hydrophobic aggregates is not known. As expected, GST alone (FIG. 5A and FIG. B, lane L) did not bind either presenilin. No other region of ubiquilin tested was able to pull-down either of the presenilins, indicating that binding of presenilin to ubiquilin is mediated by sequences spanning the UBA domain and the COOH-terminal tail. These in vitro binding assay results were consistent with the interaction data of ubiquilin recovered in the Y2H screen.

Full-length ubiquilin was expressed using the pGEM-CMV mammalian expression vector (Janicki and Monteiro 1997) (see FIG. 2 IV). In addition, two ubiquilin fusion proteins were constructed within the same expression vector. In one, green fluorescent protein (GFP) was fused to the NH₂ terminus of ubiquilin (beginning at residue 20 [Ala]), whereas in the other, a myc epitope tag was fused to the COOH terminus of full-length ubiquilin.

HeLa cells were grown at 37° C. in DME supplemented with 10% FBS and transfected with plasmid DNTAs, using the calcium phosphate coprecipitation method.

Western Blot Analysis

Cells were lysed in buffer containing protease inhibitors (Monteiro and Mical 1996), and protein concentrations were determined by the bicinchonic acid (BCA) assay. Lysates containing known amounts of protein were mixed with sample loading buffer (Janicki and Monteiro 1999) and either heated at 100° C. for 5 min or at 37° C. for 15 min before separation by SDS-PAGE. The 37° C. incubation was used to detect presenilin proteins by minimizing their tendency to aggregate and resist entry into the gels. After gel electrophoresis, proteins were transferred onto nitrocellulose filters and immunoblotted according to procedures described previously (Janicki and Monteiro 1997).

Immunoprecipitation and Cell Fractionation Studies

HeLa cells, 17 h after transfection with PS2 plasmid DNA, were collected in immunoprecipitation buffer (50 mM Tris-HCl, pH 7.5, 150 mM NaCl, 0.5% NP-40, 2 mM EDTA) and homogenized by gentle strokes with a Dounce homogenizer. After centrifugation, the pellet was discarded, and the proteins were immunoprecipitated from the remaining supernatant by adding rabbit antisera (preimmune, anti-PS2-NH₂ terminus, or anti-PS2-Loop) and protein A-Sepharose CL-4B beads (Stabler, et al. 1999). The beads were recovered by centrifugation and washed with 0.5% NP-40 in 1.0×PBS. They were boiled in sample loading buffer, and the bound proteins were analyzed by SDS-PAGE and immunoblotting. Blots were probed with primary anti-ubiquilin-B antibodies, which were subsequently detected with ¹²⁵I-protein A. Soluble, insoluble, Triton X-100-soluble, and Triton X-100-insoluble protein fractions of untransfected or PS2-transfected

HeLa cells were prepared according to the procedure described previously (Stabler, et al. 1999). Equal amounts of each fraction were separated by SDS-PAGE and, after electroblot transfer to nitrocellulose filters, were immunoblotted with either affinity-purified anti-ubiquilin-C antibody, anti-PS2-NH₂ terminus antibody, or anti-lamin A/C antibody (Mical and Monteiro 1998). The partitioning of endogenous lamin A/C proteins was monitored, since their cell fractionation behavior was known (Mical and Monteiro 1998; Stabler, et al. 1999).

To support the in vitro binding data, coimmunoprecipitation and cell fractionation experiments between PS2 and endogenous ubiquilin were also performed. PS2-transfected HeLa cell extracts were immunoprecipitated with two different anti-PS2 specific antibodies, separated by SDS-PAGE, and immunoblotted with anti-ubiquilin-B antibodies. Ubiquilin protein coimmunoprecipitated with both of the anti-PS2 antibodies, one raised against the loop and the other to the NH₂-terninal sequences, but did not coimmunoprecipitate when the preimmune sera was used (FIG. 5C). Additional verification of ubiquilin binding to PS2 was obtained in cell fractionation studies. Untransfected and PS2-transfected HeLa cells were fractionated either in the absence or presence of Triton X-100 detergent into soluble and insoluble fractions. Equivalent amounts of the fractions were then immunoblotted to determine the amount of ubiquilin protein in the two fractions. In untransfected cells, almost all of the ubiquilin protein was present in the soluble fraction, independent of detergent treatment (FIG. 5D, lanes S- and S+). This is consistent with the predicted hydrophilic nature of ubiquilin protein. However, in PS2-transfected cells, 30% more ubiquilin protein separated with the pellet fraction in the absence of detergent treatment (FIG. 5F, lane P). This change in solubility is likely a consequence of ubiquilin binding to overexpressed membrane-bound PS2, which also fractionated in the insoluble membrane-containing pellet fraction (FIG. 5E, lane P−). As expected for integral membrane proteins, Triton X-100 treatment caused the release of PS2 into the soluble fraction (FIG. 5E, lane S+), which coincided with ubiquilin once again separating completely in the soluble fraction (FIG. 5F, lane S+).

Ubiquilin Localizes to the Nucleus and Cytoplasm

To determine the intracellular distribution of ubiquilin rabbit pAb was used that was generated to two different GST-ubiquilin fusion proteins as set forth hereinafter.

Rabbit anti-ubiquilin polyclonal antisera were generated against purified GST-ubiquilin fusion proteins B or C (FIG. 2 III) by Covance Research Products, Inc. Affinity-purified anti-ubiquilin-C antibodies were obtained by passing anti-C-serum first over a column containing GST alone coupled to CH-Sepharose 4B beads at 4° C. overnight, to eliminate antibodies binding to GST, and then by mixing the flow-through fraction with ubiquilin-coupled CH-Sepharose 4B beads at 4° C. overnight. After extensive washing, antibodies that bound to the ubiquilin-coupled beads were eluted dropwise with 0.1 M sodium citrate and 0.3 M sodium chloride solution, pH 2.2, and collected as 240-μl fractions into tubes containing 40 μl 1 M Tris-HCl, pH 9.1, to restore neutral pH. In addition to fusion proteins B and C, the following primary antibodies were also used including: goat anti-PS1, anti-PS2, and anti-BiP (NH₂ terminus specific; Santa Cruz Biotechnology, Inc.); rabbit anti-PS2 (NH₂ terminus or loop specific, raised to the corresponding GST-PS2 fusion proteins) (Janicki and Monteiro 1997, Janicki and Monteiro 1999) anti-ubiquitin (Dako), anti-lamin A/C (Mical and Monteiro 1998), anti-calnexin, and anti-calreticulin (StressGen); and mouse anti-GFP (Zymed Laboratories), anti-tubulin (Sigma-Aldrich), and anti-lamin B (MatriTect). Binding of primary antibodies were detected with ¹²⁵I-protein A or by incubation with appropriate secondary horseradish peroxidase-conjugated antibodies (New England Biolabs, Inc.) using autoradiography or chemiluminescence, respectively.

Both antisera detected endogenous and overexpressed 66-kD ubiquilin polypeptides, which were not detected by the preimmune sera (FIG. 3D). Although one antiserum reacted with an unknown protein of 55 kD (FIG. 3E), this reactivity was subsequently removed by affinity purification of the antibody (FIG. 3F). Immunofluorescence staining of ubiquilin within cells was performed with the specific anti-ubiquilin antibodies. The procedure used for immunofluorescence staining of HeLa cells was described previously (Janicki and Monteiro 1997).

Indirect immunofluorescent microscopy, digital image capture, and subsequent manipulation and merging of images were done with a Leica DM IRD microscope attached to a PowerMac computer running Signal Analytics IPLab Spectrum software (Stabler, et al. 1999). Confocal images were captured on a Axiovert 100 microscope using LSM software (ZEISS). The primary antibodies used were: rabbit anti-ubiquilin-B and affinity-purified anti-ubiquilin-C, goat anti-PS1 and anti-PS2 (NH2 terminus specific) (Santa Cruz Biotechnology, Inc.), and mouse anti-myc (Mical and Monteiro 1998).

Indirect immunofluorescence, as well as laser confocal microscopy, revealed endogenous intracellular localization of ubiquilin in HeLa cells to both the nucleus and cytoplasm (FIG. 6B, FIG. 6E, and FIG. 6G). Compared with the preimmune sera (FIG. 6A and FIG. 6D), both antibodies revealed similar staining patterns (FIG. 6B and FIG. 6E). In some cells, endogenous ubiquilin staining was stronger in the nucleus (FIG. 6E, arrowheads). Furthermore, in a small subset of cells, ubiquilin within the cytoplasm was localized to punctate structures (FIG. 6B and FIG. 6E, arrows). The frequency of these structures dramatically increased upon overexpression of full-length untagged ubiquilin (FIG. 6C and FIG. 6F). To corroborate the anti-ubiquilin staining pattern, additional localization studies were performed on myc- and GFP-tagged ubiquilin proteins in HeLa cells (FIG. 2 IV, O and N, respectively). Overexpressed myc-tagged ubiquilin stained with anti-myc mAb exhibited the same intracellular structures (FIG. 6I) as overexpressed wild-type ubiquilin (FIG. 6C and FIG. 6F). Finally, the same fluorescent staining pattern was reproduced upon overexpression of GFP-ubiquilin (FIG. 6H). Since the GFP-ubiquilin image in FIG. 6H was captured from live and unfixed cells without antibodies, this established that the anti-ubiquilin ubiquilin structures seen previously were not artifacts of the paraformaldehyde fixation or immunofluorescence staining procedures.

Protein Accumulation Studies

HeLa cells were transfected with expression constructs encoding ubiquilin protein (15 μg plasmid DNA), PS2 protein (7 μg plasmid DNA), or both. At 12 h after transfection, the cells were incubated for an additional 5-6 h in DME/FBS media in the absence or presence of proteasome inhibitors (20 μM synthetic lactacystin or 40 μM MG-132; Calbiochem-Novabiochem″>Calbiochem-Novabiochem). Afterwards, the cells were lysed in buffer containing protease inhibitors and the protein concentrations were determined. Protein extracts totaling 100 tμg were loaded per lane, separated by SDS-PAGE, and immunodetected with various antibodies. In a follow up experiment, HeLa cells were cotransfected with a constant amount of PS2 (7 μg plasmid DNA) and increasing amounts of either ubiquilin (0, 1, 2, 3, and 4 μg plasmid DNA) or GFP (0, 1, 2, 3, and 4 μg plasmid DNA) expression vectors.

Since ubiquilin contains multiple ubiquitin-related structural motifs, association of ubiquilin with presenilin was investigated to determine whether it would have an effect on presenilin-protein modification and/or stability. Treatment of mock-transfected and PS2-transfected HeLa cells with proteasome inhibitors lactacystin (20 μM) or MG-132 (40 μM), as expected, increased the overall amount of ubiquitinated proteins in cells (FIG. 8A, lanes 2 and 3, 5 and 6, and 8 and 9 versus lanes 1, 4, and 7, respectively). However, these conditions did not markedly increase PS2 protein levels (FIG. 8B, lanes 4-6). Remarkably, coexpression of ubiquilin with PS2 caused a substantial (>10-fold) increase in PS2 protein accumulation (FIG. 8B, lanes 7-9) compared with overexpression of PS2 alone (FIG. 8B, lanes 46). This effect was independent of treatment with lactacystin or MG-132, as it also occurred just as robustly in cells not treated with the proteasome inhibitors. Particularly striking was the increased accumulation in SDS-PAGE gels of full-length uncleaved PS2 protein (FIG. 8B, arrowhead) and higher molecular weight complexes that PS2 forms. Increasing the amount of transfected ubiquilin plasmid DNA, whereas maintaining constant levels of transfected PS2 plasmid DNA, revealed a dose-dependent effect by ubiquilin on PS2 accumulation within the cells (FIG. 8E, lanes 1-5). This effect was specific for ubiquilin expression, since cotransfection of increasing amounts of GFP plasmid DNA with PS2 did not increase PS2 protein accumulation (FIG. 8F, lanes 1-5). The blot in FIG. 8E was exposed for a shorter time than that in FIG. 8F to optimally illustrate the subtle changes in PS2 protein levels. In these experiments, the accumulation of full-length PS2 protein was routinely observed, but not the endoproteolytic cleaved forms of the protein (Haass and De Strooper 1999). In fact, recent reports have indicated that cleavage of presenilins is not essential for biological function of the presenilins, namely by its ability to rescue PS activity in C. elegans (Jacobsen, et al. 1999; Steiner, et al. 1999). The absence of detectable endoproteolytic cleaved forms of PS2 is more clearly illustrated in FIG. 8G, which shows that an antibody specific for PS2-loop sequences downstream of the proposed endoproteolytic cleavage site recognized the same 54 kD and higher PS2 species (as the PS2 NH₂-terminal specific antibody). Interestingly, though ubiquilin caused a dose-dependent increase in presenilin accumulation, it did not alter the accumulation levels of various other endogenous HeLa proteins, such as lamin B, calreticulin, calnexin, BiP, and -tubulin (FIG. 8H). Similar experiments repeated with PS1 indicated that ubiquilin also promoted a dose-dependent increase in PS 1 protein accumulation in HeLa cells (data not shown).

Pulse-Chase Experiments

To determine if the ubiquilin-induced increase in presenilin accumulation is due to a change in the rate of presenilin protein turnover, pulse-chase experiments of HeLa cells were carried out in which PS2 protein was coexpressed with either ubiquilin or GFP (as a control). Exponentially growing HeLa cells maintained in flasks were trypsinized and resuspended at a density of 2.68×10⁶ cells/ml in Opti-MEM medium (GIBCO BRL).

Two 0.5-ml aliquots of the cell suspension were each electroporated with a mixture of either 7 μg PS2 and 15 μg of EGFP-C1 (CLONTECH Laboratories, Inc.) expression plasmid DNA or 7 μg PS2 and 15 μg of ubiquilin expression plasmid DNA. The aliquots containing the same DNA mixture were then combined, resuspended in 28 ml of OptiMEM medium (containing 10% FBS), and 2 ml of the suspension was plated in 14 separate wells (9.5 cm ²). The cells were incubated for 7 h to allow for attachment to the dishes, and then the medium was removed and replaced with DME containing 10% FBS. Incubation was continued for an additional 14 h. The cells were starved for 45 min in methionine-deficient DME containing 10% dialyzed FBS. The medium was removed and the cells in each well were pulse labeled by adding 1 ml of the methionine-deficient medium containing 150 μCi of [³⁵S]methionine (1,000 Ci/mmol; Amersham Phannacia Biotech) for 1 h. After labeling, the cells were washed twice with DME containing 1 niM nonradioactive L-methionine and 10% dialyzed FBS, and then they were chased with 2 ml of the same medium for 0-6 h. At appropriate time intervals, the cells were washed three times with ice-cold 1.0×PBS and lysed in buffer containing protease inhibitors (Monteiro and Mical 1996). The lysates were diluted 100-fold with immunoprecipitation buffer before PS2 proteins were immunoprecipitated, which was done by the addition of 5 μl rabbit anti-loop PS2 antibody (directed against GST-PS2 loop purified protein) (Janicki and Monteiro 1997) and 100 μl of a slurry of protein A-Sepharose (Amersham Pharmacia Biotech). The immunoprecipitated proteins attached to the Sepharose beads were eluted by incubation with protein sample loading buffer (lacking SDS), which contained 8 M urea, 10 mM DTT, and 10% β-mercaptoethanol (Janicki and Monteiro 1999), at 37° C. for 15 min. The entire volume of samples were separated by SDS-PAGE on 8.5% gels. After electrophoresis, the gel was stained with Coomassic blue, impregnated with 2,5-diphenyloxazole, and dried (Stabler, et al. 1999). The radioactivity of the immunoprecipitated PS2 band in each lane, corresponding to full-length PS2 protein, was quantified using a Molecular Dynamics Storm 840 Phosphoimager using ImageQuant software (Molecular Dynamics).

The half-life of endogenous HeLa ubiquilin protein was estimated by pulse labeling mock-electroporated HeLa cells for 1 h with 100 μCi of [³⁵S]methionine, followed by a chase with nonradioactive medium for 0-21 h. Ubiquilin protein was immunoprecipitated using 5 μl of rabbit anti-ubiquilin-C antibody, and the amount of radioactivity incorporated in the ubiquilin bands was determined after SDS-PAGE and phosphoimage analysis.

PS2 protein in the pulse-chase lysates was immunoprecipitated using an anti-PS2-loop antibody and resolved by SDS-PAGE (FIG. 9A). Phosphoimage analysis of the immunoprecipitated full-length PS2 band (54 kD), indicated that the half-life of PS2 protein to be approximately similar. 3.1 and 2.9 h when coexpressed with GFP or ubiquilin, respectively (FIG. 9A and FIG. 9B). Apart from the prominent PS2 band at 54 kD, a smear of bands with varying intensity was resolved after SDS-PAGE, using the PS2-loop antibody for immunoprecipitation. Some of these bands appear to be PS2 related, especially the higher molecular weight species, as they corresponded to similar PS2 immunoreactive species using this and other PS2 antibodies (FIG. 8E and FIG. 8G) (Janicki and Monteiro 1997, Janicki and Monteiro 1999). Phosphoimage analysis of the smear of bands above the 97-kD marker indicated that the rate of turnover of these larger PS2 complexes was also similar in both GFP- and ubiquilin-coexpressing cells (data not shown).

A notable difference of these pulse-labeling experiments was the increased amount of immunoprecipitated PS2-labeled proteins in cells that were overexpressing ubiquilin, compared with GFP. Despite using similar amounts of PS2 DNA for electroporation within parallel-labeling experiments, a 1.6-2.0-fold increase in PS2-labeled protein was routinely obtained when PS2 was coexpressed with ubiquilin compared with GFP. This effect was seen in three separate experiments (data not shown). Overall, these results suggest that the ubiquilin-induced elevation in presenilin accumulation does not involve a substantial change in the rate of presenilin-protein turnover, but instead may facilitate increased PS2 protein synthesis. Finally, to determine the rate of endogenous ubiquilin protein turnover, another pulse-chase experiment of mock-transfected HeLa cells was performed and immunoprecipitated the endogenous ubiquilin protein using anti-ubiquilin-C antibody (FIG. 9C). Phosphoimage analysis indicated that pulse labeled ubiquilin decays exceedingly slowly, only 15% over the 21 h chase period, from which we estimate the half-life of the protein to be 90 h.

Immunohistochemistry of Human Brain Tissue Sections Reveals Ubiquilin Immunoreactivity in Neurons as well as within Neuropathological Lesions such as Neurofibrillary Tangles and Lewy Bodies

Since neuropathological lesions in AD and PD have been found to be highly immunoreactive to ubiquitin antibodies, it was investigated whether these structures may also contain ubiquilin immunoreactivity. Hippocampus brain tissue from six AD cases (aged 77-95) and five control cases (aged 19-71) were fixed in methacarn (6:3:1, chloroform/methanol/acetic acid) overnight. Locus corculeus or substantia nigra brain tissue from three PD cases (aged 53-66) were fixed in formalin overnight, whereas frontal cortex brain tissue from two cases of diffuse Lewy body disease (aged 49 and 85) were fixed in either formalin or methacarn overnight. Samples were dehydrated and embedded in paraffin. Sections were cut 6-μm thick and mounted onto glass slides. After deparaffinization with xylene, sections were hydrated through graded ethanol treatment, and the endogenous peroxidase activity was eliminated by incubation in 3% hydrogen peroxide for 30 min. Nonspecific binding sites were blocked with 10% normal goat serum in Tris-buffered saline (50 mM Tris-HCl, 150 mM NaCl, pH 7.6) for 30 min before application of either preimmune sera, affinity-purified anti-ubiquilin-C antibody, or anti-ubiquilin-B antibody. Immunostaining was performed using the peroxidase-anti-peroxidase method, with DAB as cosubstrate (Sternberger 1986). Color images of the stained slides were captured using a Sony Digital Photo Camera DKC-5000 on a Nikon Eclipse E600 microscope using Adobe Photoshop® software.

Immunohistochemistry of adult human brain sections was performed with anti-ubiquilin-B and anti-ubiquilin-C antibodies. The former was raised against ubiquilin polypeptides that were devoid of the UB domain, whereas the latter was raised against ubiquilin polypeptides lacking the UBA domain as well (FIG. 2 III, B and C, respectively). Compared with the preimmune sera, which showed no specific staining (FIG. 10A and FIG. 10C), both antibodies strongly stained neurons with little, if any, staining of glia (FIG. 10B, FIG. 10D, and FIG. 10E). In general, the anti-ubiquilin-C antibody stained neurons with greater clarity and intensity. As with cultured cells, anti-ubiquilin staining in neurons was to the nucleus and the cytoplasm. In addition, there is clear evidence for ubiquilin staining within long axonal processes (FIG. 10D). Interestingly, neurons containing neurofibrillary tangles (NFTs) from AD affected brains seemed to stain more intensely compared with control neurons (FIGS. 10E and 10F). Moreover, examination of ubiquilin staining in brains afflicted with PD (not shown) and diffuse LB disease revealed robust staining of Lewy bodies (FIG. 10H).

Y2H interaction, GST pull-down experiments, coimmunoprecipitation studies, changes in the cellular fractionation bf proteins, and colocalization of the proteins expressed in vivo provide compelling evidence that ubiquilin and the presenilins interact with one another. Ubiquilin is an important protein because it contains multiple ubiquitin-related domains typically thought to be involved in targeting proteins for degradation, yet ubiquilin promotes increased presenilin protein accumulation. Moreover, ubiquilin is highly expressed in neurons of human brain and is associated with NFTs and Lewy bodies of AD and PD brains, respectively.

The promotion of presenilin protein accumulation by ubiquilin overexpression is noteworthy as a new means by which presenilin levels may be modulated. By conducting pulse-chase experiments, it was found that ubiquilin induced an increase in presenilin protein maturation, but did not dramatically affect presenilin protein turnover. The net effect over time from increased presenilin protein synthesis and undisturbed turnover rate would eventually lead to an elevation of the intracellular pool of presenilin proteins.

The region in ubiquilin that binds presenilins was mapped to the COOH-terminal region containing the UBA domain, which is highly conserved in ubiquilin family members from human to yeast (FIG. 4). This sequence was both necessary and sufficient to bind PS1 and PS2 in vitro. The UBA domain is a recently recognized motif present in one or two copies in a variety of proteins involved in the ubiquitin/proteasome folding/degradation pathway, UV excision repair, and phosphorylation (Hofmann and Bucher 1996). The UBA domain consists of 55 total residues of which 45 appear to be core residues. 15 of the core residues are extremely well conserved, suggesting they may be important for determining the overall structure of the domain. Through nuclear magnetic resonance, the structure of the COOH-terminal UBA domain of the human DNA repair protein HHR23A (RAD23 homologue) has been determined. It is a compact three-helix bundle with an exposed hydrophobic surface predicted to be involved in protein-protein interactions (Dieckmann, et al. 1998). The strong binding of the ubiquilin UBA domain to presenilins may be facilitated by similar protein-protein interactions.

Using a combination of immunological and GFP-tagging approaches, it has been demonstrated herein that ubiquilin is localized to both the nucleus and the cytoplasm in HeLa cells. The intensity of nuclear staining was variable, with some nuclei staining very brightly. The variability may be related to cell cycle changes of cyclin A (a protein to which ubiquilin's homologue XDRP1 has been shown to interact) levels within the nucleus. The cytoplasmic distribution of ubiquilin was also variable. In untransfected cells, endogenous ubiquilin had a fine punctate appearance, with hints of association to a network-like pattern, possibly the ER. In some of these cells, ubiquilin accumulated in larger spherical structures throughout the cytoplasm, whose number and size were variable. Upon ubiquilin overexpression, the cells formed even larger and more numerous ubiquilin-containing structures. Under confocal microscopy, the ubiquilin-staining pattern colocalized almost perfectly with the presenilin-staining pattern.

In summary, ubiquilin is the first presenilin-interacting protein that has been found to regulate presenilin levels in cells.

### REFERENCES

1. Biggins, S., lvanovska, I., and Rose, M. D. 1996. Yeast ubiquitin-like genes are involved in duplication of the microtubule-organizing center. J Cell Biol. 133:1331-1346.

2. Braun, B. C., Glickman, M., Kraft, R., Dahlmann, B., Kloetzel, P. M., Finley, D., and Schmidt, M. 1999. The base of the proteasome regulatory particle exhibits chaperone-like activity. Nat. Cell Biol. 1:221-226.

3. Cotman, C. W. 1998. Apoptosis decision cascades and neuronal degeneration in Alzheimer's disease. Neurobiol. Aging 19:S29-S32.

4. Davis, J. A., Naruse, S., Chen, H., Eckman, C., Younkin, S., Price, D. L., Borchelt, D. R., Sisodia, S. S., and Wong, P. C. 1998. An Alzheimer's disease-linked PS1 variant rescues the developmental abnormalities of PS1-deficient embryos. Neuron 20:603-609.

5. Dieckmann, T., Withers-Ward, E. S., Jarosinski, M. A., Liu, C. F., Chen, I. S., and Feigon, J. 1998. Structure of a human DNA repair protein UBA domain that interacts with HIV-1 Vpr. Nat. Struct. Biol 5:1042-1047.

6. Donoviel, D. B., Hadjantonakis, A. K., Ikeda, M., Zheng, H., Hyslop, P. S., and Bernstein, A. 1999. Mice lacking both presenilin genes exhibit early embryonic patterning defects. Genes Dev 13:2801-2810.

7. Fraser, P. E., Levesque, G., Yu, G., Mills, L. R., Thirlwell, J., Frantseva, M., Gandy, S. E., Seeger, M., Carlen, P. L., and St George-Hyslop, P. 1998. Presenilin-1 is actively degraded by the 26S proteasome. Neurobiol. Aging 19:S19-S21.

8. Funakoshi, M., Geley, S., Hunt, T., Nishimoto, T., and Kobayashi, H. 1999. Identification of XDRP 1; a Xenopus protein related to yeast Dsk2p binds to the N-terminus of cyclin A and inhibits its degradation. EMBO (Eur. Mol. BioL Organ.) J 18:5009-5018.

9. Garcia-Mata, R., Bebok, Z., Sorscher, E. J., and Sztul, E. S. 1999. Characterization and dynamics of aggresome formation by a cytosolic GFP-chimera. J. Cell Biol. 146:1239-1254.

10. Georgakopoulos, A., Marambaud, P., Efthimiopoulos, S., Shioi, J., Cui, W., Li, H. C., Schutte, M., Gordon, R., Holstein, G. R., Martinelli, G., Mehta, P., Friedrich, V. L., Jr., and Robakis, N. K. 1999. Presenilin-1 forms complexes with the cadherin/catenin cell-cell adhesion system and is recruited to intercellular and synaptic contacts. Mol. Cell 4:893-902.

11. Guo, Q., Sopher, B. L., Furukawa, K., Pham, D. G., Robinson, N., Martin, G. M., and Mattson, M .P. 1997. Alzheimer's presenilin mutation sensitizes neural cells to apoptosis induced by trophic factor withdrawal and amyloid b-peptide: involvement of calcium and oxyradicals. J Neurosci 17:4212-4222.

12. Haass, C., and De Strooper, B. 1999. The presenilins in Alzheimer's disease-proteolysis holds the key. Science 286:916-919.

13. Hardy, J. 1997. Amyloid, the presenilins and Alzheimer's disease. Trends Neurosci 20:154-159.

14. Herreman, A., Hartmann, D., Annaert, W., Saftig, P., Craessaerts, K., Semeels, L., Umans, L., Schrijvers, V., Checler, F., and Vanderstichele, H. et al. 1999. Presenilin-2 deficiency causes a mild pulmonary phenotype and no changes in amyloid precursor protein processing but enhances the embryonic lethal phenotype of presenilin-1 deficiency. Proc. Natl. Acad. Sci. USA 96:11872-11877.

15. Hofmann, K., and Bucher, P. 1996. The UBA domain: a sequence motif present in multiple enzyme classes of the ubiquitination pathway. Trends Biochem. Sci 21:172-173.

16. Honda, T., Yasutake, K., Nihonmatsu, N., Mercken, M., Takahashi, H., Murayama, O., Murayama, M., Sato, K., Omori, A., Tsubuki, S., Saido, T.C., and Takashima, A. 1999. Dual roles of proteasome in the metabolism of presenilin-1. J. Neurochem 72:255-261.

17. Jacobsen, H., Reinhardt, D., Brockhaus, M., Bur, D., Kocyba, C., Kurt, H., Grim, M. G., Baumeister, R., and Loetscher, H. 1999. The influence of endoproteolytic processing of familial Alzheimer's disease presenilin 2 on Aβ42 amyloid peptide formation. J. Biol. Chem. 274:35233-35239.

18. Janicki, S., and Monteiro, M. J. 1997. increased apoptosis arising from increased expression of the Alzheimer's disease-associated presenilin-2 mutation (N141I). J . Cell Biol 139:485-495.

19. Janicki, S. M., and Monteiro, M. J. 1999. Presenilin overexpression arrests cells in the G1 phase of the cell cycle. Arrest potentiated by the Alzheimer's disease PS2(N141I) mutant. Am. J. Pathol 155:135-144.

20. Johnston, J. A., Ward, C. L., and Kopito, R. R. 1998. Aggresomes: a cellular response to misfolded proteins. J. Cell Biol 143:1883-1898.

21. Katayama, T., Imaizumi, K., Sato, N., Miyoshi, K., Kudo, T., Hitomi, J., Morihara, T., Yoneda, T., Gomi, F., and Mori, Y. et al. 1999. Presenilin-1 mutations downregulate the signalling pathway of the unfolded-protein response. Nat. Cell Biol 1:479-485.

22. Kaye, F. J., Modi, S., Ivanovska, I., Koonin, E. V., Thress, K., Kubo, A., Kombluth, S., and Rose, M. D. 2000. A family of ubiquitin-like proteins binds the ATPase domain of Hsp70-like Stch. FEBS Leli 467:348-355.

23. Kehoe, P., Wavrant-De Vrieze, F., Crook, R., Wu, W. S., Holmans, P., Fenton, I., Spurlock, G., Norton, N., Williams, H., and Williams, N. et al. 1999. A full genome scan for late onset Alzheimer's disease. Hum. Mol. Genet 8:237-245.

24. Kim, T. W., Pettingell, W. H., Hallmark, O. G., Moir, R. D., Wasco, W., and Tanzi, R. E. 1997. Endoproteolytic cleavage and proteasomal degradation of presenilin-2 in transfected cells. J Biol. Chem 272:11006-11010.

25. Kleijnen, M. F., Shih, A. H., Zhou, P., Kumar, S., Soccio, R. E., Kedersha, N. L., Gill, G., and Howley, P. M. 2000. The hPLIC proteins may provide a link between the ubiquitination machinery and the proteasome. Mol. Cell 6:409-419.

26. Kovacs, D.M., Fausett, H. J., Page, K. J., Kim, T. W., Moir, R. D., Merriam, D. E., Hollister, R. D., Hallmark, O. G., Mancini, R., and Felsenstein, K. M. et al. 1996. Alzheimer-associated presenilins 1 and 2: neuronal expression in brain and localization to intracellular membranes in mammalian cells. Nat. Med 2:224-229.

27. Krobitsch, S., and Lindquist, S. 2000. Aggregation of huntingtin in yeast varies with the length of the polyglutamine expansion and the expression of chaperone proteins. Proc. Natl. Acad. Sci. USA. 97:1589-1594.

28. Leissring, M. A., Parker, I., and LaFerla, F. M. 1999. Presenilin-2 mutations modulate amplitude and kinetics of inositol 1,4,5-trisphosphate-mediated calcium signals. J Biol. Chem 274:32535-32538.

29. Levitan, D., and Greenwald, 1. 1995. Facilitation of lin-12-mediated signalling by sel-12, a Caenorhabditis elegansS182 Alzheimer's disease gene. Nature 377:351- 354.

30. Mattson, M. P., Guo, Q., Furukawa, K., and Pedersen, W. A. 1998. Presenilins, the endoplasmic reticulum, and neuronal apoptosis in Alzheimer's disease. J Neurochem 70:1-14.

31. Mical, T. I., and Monteiro, M. J. 1998. The role of sequences unique to nuclear intermediate filaments in the targeting and assembly of human lamin B: evidence for lack of interaction of lamin B with its putative receptor. J Cell Sci 111:3471-3485.

32. Monteiro, M. J., and Mical, T. I. 1996. Resolution of kinase activities during the HeLa cell cycle: identification of kinases with cyclic activities. Exp. Cell Res 223:443-451.

33. Mori, H., Kondo, J., and Ihara, Y. 1987. Ubiquitin is a component of paired helical filaments in Alzheimer's disease. Science 235:1641-1644.

34. Niwa, M., Sidrauski, C., Kaufinan, R. J., and Walter, P. 1999. A role for presenilin-1 in nuclear accumulation of Irel fragments and induction of the mammalian unfolded protein response. Cell 99:691-702.

35. Perry, G., Friedman, R., Shaw, G., and Chau, V. 1987. Ubiquitin is detected in neurofibrillary tangles and senile plaque neurites of Alzheimer disease brains. Proc. Natl. Acad. Sci. USA 84:3033-3036.

36. Pickart, C.M. 1998. Polyubiquitin chains. In Peters J.-M., Harris J. R., Finley D., eds. Ubiquitin and the Biology of the Cell. New York, Plenum Press, 19-63.

37. Price, D. L., Tanzi, R. E., Borchelt, D. R., and Sisodia, S. S. 1998. Alzheimer's disease: genetic studies and transgenic models. Annu. Rev. Genet 32:461-493.

38. Qian, S., Jiang, P., Guan, X. M., Singh, G., Trumbauer, M. E., Yu, H., Chen, H. Y., Van de Ploeg, L. H., and Zheng, H. 1998. Mutant human presenilin-1 protects presenilin-1 null mouse against embryonic lethality and elevates Abl-42/43 expression. Neuron 20:611-617.

39. Raina, A. K., Monteiro, M. J., McShea, A., and Smith, M. A. 1999. The role of cell cycle-mediated events in Alzheimer's disease. Int. J Exp. Pathol 80:71-76.

40. Shen, J., Bronson, R. T., Chen, D. F., Xia, W., Selkoe, D. J., and Tonegawa, S. 1997. Skeletal and CNS defects in presenilin-1-deficient mice. Cell 89:629-639.

41. Stabler, S. M., Ostrowski, L. L., Janicki, S. M., and Monteiro, M. J. 1999. A myristoylated calcium-binding protein that preferentially interacts with the Alzheimer's disease presenilin-2 protein. J Cell Biol. 145:1277-1292.

42. Steiner, H., Romig, H., Grim, M. G., Philipps, U., Pesold, B., Citron, M., Baumeister, R., and Haass, C. 1999. The biological and pathological function of the presenilin-1 exon 9 mutation is independent of its defect to undergo proteolytic processing. J Biol. Chem. 274:7615-7618.

43. Stemberger, L. A. 1986. Immunochemistry. 3rd ed New York, John Wiley & Sons, Inc, pp. 524.

44. Struhl, G., and Greenwald, 1. 1999. Presenilin is required for activity and nuclear access of Notch in Drosophila. Nature 398:522-525.

45. Warrick, J. M., Chan, H. Y., Gray-Board, G. L., Chai, Y., Paulson, H. L., and Bonini, N. M. 1999. Suppression of polyglutamine-mediated neurodegeneration in Drosophila by the molecular chaperone HSP70. Nat. Genet. 23:425-428.

46. Wong, P. C., Zheng, H., Chen, H., Becher, M. W., Sirinathsinghji, D. J., Trumbauer, M. E., Chen, H. Y., Price, D. L., Van der Ploeg, L. H., and Sisodia, S. S. 1997. Presenilin-11 is required for Notchl and DIIl expression in the paraxial mesoderm. Nature 387:288-292.

47. Wu, A. L., Wang, J., Zheleznyak, A., and Brown, E. J. 1999. Ubiquitin-related proteins regulate interaction of vimentin intermediate filaments with the plasma membrane. Mol. Cell 4:619-625.

48. Ye, Y., Lukinova, N., and Fortini, M. E. 1999. Neurogenic phenotypes and altered Notch processing in Drosophila presenilin mutants. Nature 398:525-529.

That which is claimed is:

1. A purified and isolated nucleic acid molecule encoding a protein that modulates presenilin protein levels, said protein having the amino acid sequence of SEQ ID NO: 2.

2. The purified and isolated nucleic acid molecule encoding a protein of claim 1, further comprising an amino acid sequence of SEQ ID NO. 5.

3. An isolated and purified nucleic acid molecule having the nucleic acid sequence of SEQ ID NO: 1.

4. The isolated and purified nucleic acid molecule of claim 3, wherein the nucleic acid sequence further comprises SEQ ID NO. 7.

5. An expression vector for transforming a mammalian tissue cell to express effective amounts of ubiquilin, the expression vector comprising a nucleotide sequence that encodes for the ubiquilin having an amino acid sequence of SEQ ID NO: 2.

6. The expression vector of claim 5, further comprising a nucleotide sequence that encodes for a presenilin having an amino acid sequence of SEQ ID NO. 5 or SEQ ID NO. 6.

7. An expression vector for transforming a mammalian tissue cell to coexpress effective amounts of ubiquilin and presenilin, the expression vector comprising a nucleotide sequence that encodes for the ubiquilin having an amino acid sequence of SEQ ID NO: 2 or SEQ ID NO. 4 and a nucleotide sequence that encodes for a presenilin having an amino acid sequence of SEQ ID NO: 5 or SEQ ID NO. 6.

8. The expression vector of claim 6, wherein the expression vector is delivered to the cells by a delivery modality selected from the group consisting of: vaccinia virus, adeno associated virus, retrovirus, liposome transport, and neuraltropic virus.

9. A recombinant host cell transfected with the expression vector of claim 6.

10. A recombinant host cell transfected with the expression vector of claim 7.

11. A method for producing a polypeptide comprising an amino acid sequence of SEQ ID NO: 2, the method comprising:
(a) culturing a host cell containing an expression vector containing a polynucleotide sequence of SEQ ID NO: 1; and
(b) recovering the expressed polypeptide from the host cell.

12. A method for inducing an increase in a presenilin protein maturation by protein interaction with ubiquilin, the method comprising:
introducing an expression vector to a host cell that expresses a presenilin protein, to yield a transformed host cell, wherein the expression vector comprises a nucleotide sequence encoding an amino acid sequence of SEQ ID NO. 2 or SEQ ID NO. 4;
maintaining the transformed host cell under biological conditions sufficient for expression and accumulation of the ubiquilin in the host cell; and
measuring the increase of the presenilin protein in the host cell.

13. An immunoassay to determine the presence of ubiquilin and presenilin in brain tissue, wherein the immunoassay utilizes an antibody against ubiquilin, the method comprising:
contacting sample brain tissue with an antibody that specifically binds to the ubiquilin, to form a mixture of antigen-antibody reaction products;
detecting the presence of the antigen-antibody reaction products; and
correlating the detected antigen-antibody reaction products with a control standard to show presence of ubiquilin and presenilin in the brain tissue.

14. A method to modulate presenilin protein levels by protein-protein interaction with ubiquilin, the method comprising:
interacting a presenilin peptide comprising an amino acid sequence of SEQ ID NO. 5 or SEQ ID NO 6 with a ubiquilin protein of SEQ ID NO. 2 or SEQ ID NO. 4.

15. A method of quantifying neurofibrillary tangles and Lewy bodies in Alzheimer's disease and Parkinson's disease affected brains, the method comprising:
contacting sample brain tissue with an anti-ubiquilin antibody that specifically binds to the ubiquilin, to form a mixture of antigen-antibody reaction products;
detecting the presence of the antigen-antibody reaction products; and
correlating the detected antigen-antibody reaction products with a control standard to quantify neurofibrillary tangles and Lewy bodies in the brain tissue.

16. The method of claim 15, wherein the anti-ubiquilin antibody is detected by an indicator affixed to the antibody.

17. The method of claim 16, wherein the indicator is selected from the group consisting of: radioactive labels, a second antibody and horse radish peroxidase.

18. A method for treating a disorder associated with decreased presenillin synthesis, the method comprising:
administering to a subject in need of such treatment an effective amount of a pharmaceutical composition comprising an amino acid sequence of SEQ ID NO. 2.

20. A purified antibody that binds to a polypeptide comprising an amino acid sequence of SEQ ID NO: 2.
